(19) United States
(12) Patent Application Publication
(10) Pub. No.: US 2024/0326380 A1
(43) Pub. Date: Oct. 3, 2024

(54) Coacervate Compositions, Multifunctional Coatings, and Tie Layers for Recyclable and Active Barrier Films

(71) Applicant: The University of Toledo, Toledo, OH (US)

(72) Inventors: Yakov Lapitsky, Toledo, OH (US); Maria R. Coleman, Toledo, OH (US); Joseph G. Lawrence, Toledo, OH (US); Jose C. Benalcazar Bassante, Toledo, OH (US)

(73) Assignee: The University of Toledo, Toledo, OH (US)

(21) Appl. No.: 18/615,152

(22) Filed: Mar. 25, 2024

Related U.S. Application Data
(60) Provisional application No. 63/455,084, filed on Mar. 28, 2023.

(51) Int. Cl. B32B 7/10; B32B 27/08; B32B 27/32; B32B 27/36; B32B 43/00

(52) U.S. Cl. CPC B32B 7/10; B32B 27/08; B32B 27/32; B32B 27/36; B32B 43/006; B32B 2307/308; B32B 2307/7244; B32B 2553/00

(57) ABSTRACT

Coacervate compositions, tie layers, coatings, moldable viscoelastic materials, sustained release materials, kits for making coacervate compositions, and methods of using and making the same are described. A coacervate composition may include a complex between a surfactant and a polyelectrolyte in a solvent, where the surfactant includes a cis double bond.

### RELATED APPLICATIONS

This application claims priority to U.S. Provisional Application No. 63/455,084 filed under 35 U.S.C. § 111 (b) on Mar. 28, 2023, the disclosure of which is incorporated herein by reference in its entirety for all purposes.

### STATEMENT REGARDING FEDERALLY SPONSORED RESEARCH

This invention was made with no government support. The government has no rights in this invention.

### BACKGROUND

The recycling of multilayer packaging materials is a challenge. Incineration does not fully recover the energy used during plastic production. The selective dissolution and precipitation is challenging when there are more than a few distinct layer types, and it is hard to delaminate many different layers in a single process. There is a need in the art for new and improved multilayer packaging materials with enhanced recyclability.

### SUMMARY

Provided is a coacervate composition comprising a complex between a polyelectrolyte and a surfactant, and a solvent, wherein the surfactant comprises a cis double bond and a hydrophobic tail, and wherein the complex is moldable, adhesive, viscoelastic, or a viscous liquid having a viscosity that exceeds a viscosity of the solvent. In certain embodiments, the surfactant includes two or more hydrophobic tails. In certain embodiments, the cis double bond is a non-terminal double bond.

In certain embodiments, the surfactant is a fatty acid. In particular embodiments, the surfactant is an unsaturated fatty acid. In certain embodiments, the polyelectrolyte comprises polyallylamine (PAH). In certain embodiments, the polyelectrolyte comprises polyallylamine (PAH) and the surfactant comprises an unsaturated fatty acid. In certain embodiments, the polyelectrolyte comprises polyallylamine (PAH) and the surfactant includes only one double bond. In certain embodiments, the polyelectrolyte comprises polyallylamine (PAH) and the surfactant comprises a fatty acid having two or more double bonds. In certain embodiments, the polyelectrolyte comprises polyallylamine (PAH) and the surfactant comprises linoleic acid. In certain embodiments, the polyelectrolyte comprises polyallylamine (PAH) and the surfactant comprises oleic acid. In certain embodiments, the polyelectrolyte comprises polyallylamine (PAH), the surfactant comprises an unsaturated fatty acid, and the solvent comprises water, ethanol, methanol, or isopropanol. In certain embodiments, the polyelectrolyte comprises polyallylamine (PAH), and the surfactant comprises oleic acid. In particular embodiments, the solvent comprises water or ethanol. In certain embodiments, the solvent is removed from the coacervates to transform them into hardened solids.

In certain embodiments, the polyelectrolyte comprises polyallylamine (PAH), and the surfactant comprises linoleic acid. In particular embodiments, the solvent comprises water or ethanol. In particular embodiments, the PAH is present in an amount ranging from about 10 wt % to about 20 wt %. In particular embodiments, the unsaturated fatty acid is present in an amount of up to about 90 wt %. In particular embodiments, the PAH and the unsaturated fatty acid have a stoichiometric ratio of about 1:1.

In certain embodiments, the coacervate composition further comprises an electrolyte soluble in the solvent. In particular embodiments, the electrolyte is NaNO₃. In particular embodiments, the electrolyte is NaCl. In particular embodiments, the electrolyte is an alkali metal salt, a halide salt, a nitrate, an ammonium salt, a sulfate, or a phosphate. In particular embodiments, the coacervate composition is in the form of a uniform film.

In certain embodiments, the coacervate composition further comprises a payload, wherein the coacervate composition is configured to release the payload over time. In particular embodiments, the payload comprises a drug, a disinfectant, a fragrance, or a dye.

In certain embodiments, the coacervate composition is in the form of a homogeneous film. In particular embodiments, the homogeneous film is disposed on a plastic support.

In certain embodiments, the coacervate composition is in the form of a bulk fluid or moldable putty-like material. In certain embodiments, the coacervate composition is in the form of a moldable, viscoelastic solid. In certain embodiments, the coacervate composition comprises macroscopic liquid coacervates.

In certain embodiments, the coacervate composition further comprises one or more additional surfactants. In certain embodiments, the coacervate composition further comprises an oil.

In certain embodiments, the solvent is water, and the coacervate composition is a moldable, viscoelastic solid.

Further provided is an article coated with a coacervate composition described herein, wherein the coacervate composition is configured to release a payload over time.

Further provided is a multilayer packaging material comprising a first plastic layer comprising a first plastic material; a tie layer comprising the coacervate composition described herein; and a second plastic layer comprising a second plastic material; wherein the tie layer is laminated together with the first plastic layer and the second plastic layer. In certain embodiments, the first plastic material or the second plastic material comprises poly(ethylene terephthalate) (PET), low-density polyethylene (LDPE), high-density polyethylene (HDPE), polypropylene (PP), ethylene-vinyl alcohol copolymer (EVOH), nylon, or poly(ethylene-vinyl acetate) (PEVA).

In certain embodiments, the tie layer comprises a payload for sustained release.

In certain embodiments, the multilayer packaging material further comprises a plurality of additional layers. In certain embodiments, the multilayer packaging material further comprises a second tie layer and a third plastic layer comprising a third plastic material, wherein the second tie layer is between the second plastic layer and the third plastic layer. In particular embodiments, the second tie layer comprises PET, LDPE, HDPE, PP, EVOH, nylon, or PEVA. In certain embodiments, the second tie layer is identical in composition to the first tie layer. In certain embodiments, the multilayer packaging material further comprises a fourth plastic layer. In certain embodiments, the multilayer packaging material further comprises a fifth plastic layer.

Further provided is a multilayer packaging material comprising a first plastic layer comprising a first plastic material; a first tie layer comprising a first coacervate composition, wherein the first coacervate composition comprises a first complex between a first polyelectrolyte and a first surfactant having a cis double bond; a second plastic layer comprising a second plastic material, wherein the first tic layer connects the first plastic layer and the second plastic layer; a second tie layer comprising a second coacervate composition, wherein the second coacervate composition comprises a second complex between a second polyelectrolyte and a second surfactant having a cis double bond; and a third plastic layer comprising a third plastic material, wherein the second tie layer connects the second plastic layer and the third plastic layer. In certain embodiments, the first surfactant has a hydrophobic tail. In certain embodiments, the second surfactant has a hydrophobic tail.

In certain embodiments, the multilayer packaging material further comprises a plurality of additional layers. In certain embodiments, the first polyelectrolyte and the second polyelectrolyte are the same. In certain embodiments, the first polyelectrolyte and the second polyelectrolyte are different. In certain embodiments, the first surfactant and the second surfactant are the same. In certain embodiments, the first surfactant and the second surfactant are different. In certain embodiments, one or both of the first polyelectrolyte and the second polyelectrolyte comprises PAH, and one or both of the first surfactant and the second surfactant comprises oleic acid or linoleic acid.

Further provided is a method of recycling a multilayer packaging material, the method comprising dissolving the tie layer of the multilayer packaging material described herein in a basic solution at a temperature and for a period of time sufficient to completely dissolve the tie layer. In certain embodiments, the basic solution has a room-temperature pH of at least about 11. Depending on the temperature and base polymer used, however, tie layer dissociation can be achieved at an even lower pH level. Accordingly, in certain embodiments, the basic solution has a room-temperature pH of at least about 10.

Further provided is a method for making a coacervate composition, the method comprising forming a complex between a polyelectrolyte and a surfactant having a cis double bond and a hydrophobic tail; and solvating the complex in a solvent to form a coacervate composition, wherein the complex is moldable, adhesive, viscoelastic, or a viscous liquid (with a viscosity higher than that of its solvent). In certain embodiments, the solvent is water or an alcohol. In particular embodiments, the alcohol is ethanol, methanol, or propanol. In certain embodiments, the solvent is a mixture of water and one or more alcohols. In certain embodiments, the method further comprising removing the solvent.

Further provided is a kit for making a coacervate composition, the kit comprising a first container housing a surfactant having a cis double bond and a hydrophobic tail; and a second container housing a polyelectrolyte. In certain embodiments, the kit further comprises a third container housing a solvent.

Further provided is a kit for making a coacervate composition, the kit comprising a first container housing a polyelectrolyte, and a second container housing a mixture of a solvent and a surfactant having a cis double bond and a hydrophobic tail.

Further provided is a kit for making a coacervate composition, the kit comprising a first container housing a surfactant having a cis double bond and a hydrophobic tail, and a second container housing a mixture of a solvent and a polyelectrolyte.

Further provided is a kit for making a coacervate composition, the kit comprising a first container housing a first mixture of a solvent and a polyelectrolyte, and a second container housing a second mixture of the solvent and a surfactant having a cis double bond and a hydrophobic tail.

Further provided is a composition comprising a moldable, viscoelastic solid, coating, or adhesive film formed through polyelectrolyte complexation of a surfactant having a non-terminal double bond-containing hydrophobic tail.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Illustration of the formation of a coacervate and a supernatant phase from the mixture of a polyelectrolyte and a surfactant.

FIG. 2: Illustration of coacervates formed through spontaneous self-assembly followed by further solvent addition/exchange.

FIG. 3: Illustration of a non-limiting example embodiment of a multilayer packaging material.

FIG. 4: Illustration of another non-limiting example embodiment of a multilayer packaging material.

FIG. 5: Illustration showing the replacement of conventional tie layers in a multilayer packaging material with a polyelectrolyte/surfactant mixture.

FIGS. 6A-6D: Photographs of PAH/fatty acid coacervates solvated in deionized water (FIGS. 6A, 6B) and ethanol (FIGS. 6C, 6D) and formed using either oleic acid (FIGS. 6A, 6C) or linoleic acid (FIGS. 6B, 6D).

FIGS. 7A-7B: Dynamic rheology of PAH/oleate (FIG. 7A) and PAH/linoleate (FIG. 7B) coacervates prepared at a 1:1 fatty acid: PAH monomer molar ratio showing the storage moduli (closed symbols), G′, and loss moduli (open symbols), G″, achieved upon swelling the coacervates in water (squares) and ethanol (circles).

FIGS. 8A-8B: Coalescence of ethanolic linoleic acid/PAH colloidal coacervates into macroscopic coacervate phases (FIG. 8A) and uniform, surface-deposited films (FIG. 8B) by 0.1 mM NaNO₃ solutions. FIG. 8A shows photographs comparing the coalescence behavior of colloidal fatty acid/PAH coacervate dispersions prepared without NaNO₃ and to those with 0.1 mM NaNO₃. FIG. 8B shows an illustration and a photograph showing that the controlled coalescence of colloidal coacervate dispersions generates uniform tie layer films.

FIG. 9: On-demand dissociation of PAH/linoleate complex-bonded multilayer film composed of PET (clear layer on the left), a PAH/linoleate tie layer (thin layer in the middle), and LDPE (opaque layer on the right). FIG. 9 shows photographs of the delamination of PET/coacervate/LDPE multilayer disks (prepared from macroscopic coacervates) triggered by the dissociation of the coacervate tie layer in 1000 mM NaOH solution at 100° C.

FIGS. 10A-10E: Molecular structure of Rhodamine B (RhB) (FIG. 10A), RhB encapsulated within PAH/oleate coacervate samples (FIG. 10B), RhB slowly diffusing into PAH/oleate samples over 6 weeks (FIG. 10C), flotation of the RhB-loaded PAH/oleate coacervate samples after centrifugation revealing these coacervate phases to (contrary to conventional ones) be the less dense of the two phases forming upon complex coacervation (FIG. 10D), and graph showing the release of the RhB dye over time (FIG. 10E).

FIG. 11: Color change in a dried PAH/linoleate complex over 3 days caused by its chemical reaction with oxygen at 40° C.

FIGS. 12A-12B: Graphs showing the dissociation kinetics of square PET/LDPE bilayer film fragments (either 4.0, 5.7, or 7.1 mm in size) tied together with PAH/oleate (FIG. 12A) or PAH/linoleate (FIG. 12B) complexes in 1 M NaOH at 100° C. The graphs show the delamination of variably sized square PET/coacervate/LDPE multilayer fragments prepared through colloidal deposition of PAH/oleate (FIG. 12A) and PAH/linoleate (FIG. 12B) coacervates in 1000 mM NaOH solution at 100° C. as a function of time.

FIG. 13: Small-angle x-ray scattering (SAXS) results showing that the liquid properties of ethanolic coacervates coincide with a transition to a less-ordered microstructure.

FIG. 14: Polarized optical microscopy results showing aqueous samples to be birefringent and ethanolic samples to be nonbirefringent.

FIG. 15: Thermogravimetric analysis (TGA) graphs showing the thermal stability of the tie layers.

FIG. 16: Graphs showing the adhesion strength of linoleic acid/PAH and oleic acid/PAH tie layers, using macroscopic aqueous coacervates and macroscopic ethanolic coacervates, in a PET/tie layer/LDPE structure, shown in the photograph on the right.

FIG. 17: Photographs showing the difficulty in applying a uniform coating of the tie layer material when it is applied as a macroscopic coacervate.

FIG. 18: Photographs of a microscopic coacervation (left) and a macroscopic coacervation (right).

FIG. 19: Photographs showing improvement is coating uniformity with a colloidal coacervate compared to a macroscopic coacervate.

FIG. 20: Graph showing adhesion strength of colloidal versus macroscopic coacervates.

FIG. 21: Photograph of experimental setup (left) and graph (right) showing the oxygen scavenging properties of a linoleic acid/PAH coacervate, which are compared to an oleic acid/PAH coacervate control.

FIG. 22: Oxygen permeance through: single-layer PET films, PET/LDPE film bilayers, and PET/coacervate/LDPE multilayers prepared using PAH/linoleate and PAH/oleate tie-layers (mean±SD). The letters (A-C) represent statistically significant differences (p<0.05) based on a Student's t-test.

FIG. 23: Photographs of coacervates formed using the ionic/nonionic surfactant mixture between PDADMAC/TX100 with SDS, having a Y^{b} of 0.35 (left) and 0.37 (right).

FIG. 24: T-peeling adhesion strength of the ethanolic PAH/linoleate coacervate tie layer prepared at the 1:1 linoleic acid: PAH amine group molar ratio and applied using colloidal dispersion deposition in LDPE/tie layer/EVOH, PET/tie layer/LDPE, and PET/tie layer/EVOH multilayers compared with those achieved with commercial tie layers (mean±SD). The commercial tie layer data was obtained from Mitsui Chemical America, DuPont, and LyondellBasell.

FIGS. 25A-25B: Representative images of differences between the colloidal dispersion deposition/drying process on a PET film achieved with PAH/oleate (FIG. 25A) and PAH/linoleate coacervates (FIG. 25B) prepared at the 1:1 fatty acid: PAH amine group molar ratio during the lamination process (where the partly dried coacervate layer is covered and compressed with a second, LDPE film).

FIGS. 26A-26C: Representative photographs showing the effect of room-temperature 1000 mM NaOH solution on (i) dry PAH/oleate and (ii) PAH/linoleate coacervates (FIG. 26A), and the times required for these coacervates to disintegrate in 1-1000 mM NaOH solution at 100° C. (mean±SD) (FIG. 26B). FIG. 26C shows the delamination time requirements of square PET/coacervate/LDPE multilayer fragments (prepared through colloidal deposition and with 4.0-mm dimensions) in 0.1-1000 mM NaOH solutions at 100° C. (mean±SD). The lines are guides to the eye.

FIGS. 27A-27C: Photographs demonstrating the solubility/dispersibility of 0.05 wt % oleic acid (FIG. 27A), linoleic acid (FIG. 27B), and PAH (FIG. 27C) in room temperature (i, ii) and 100° C. (iii, iv) 10 mM (i, iii) and 1000 mM (ii, iv) NaOH solution.

FIGS. 28A-28B: Delamination of the PET/coacervate/LDPE multilayer disks laminated with PAH/oleate (FIG. 28A) and PAH/linoleate (FIG. 28B) macroscopic coacervates in 1000 mM NaOH solution at 100° C. as a function of time.

FIG. 29: Delamination time of 4.0-mm square fragments of PET/coacervate/LDPE, LDPE/coacervate/EVOH, PET/coacervate/EVOH, and PET/coacervate/EVOH/coacervate/LDPE multilayers in 1000 mM NaOH solution at 100° C. prepared through the colloidal dispersion deposition of PAH/linoleate coacervate, as a function of time.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

In accordance with the present disclosure, coacervate compositions involving a polyelectrolyte and a surfactant can be utilized as tie layers for multilayer packaging materials, may have controlled payload release and oxygen scavenging properties, and may be easily dissolved on demand and recycled under conventional recycling process conditions.

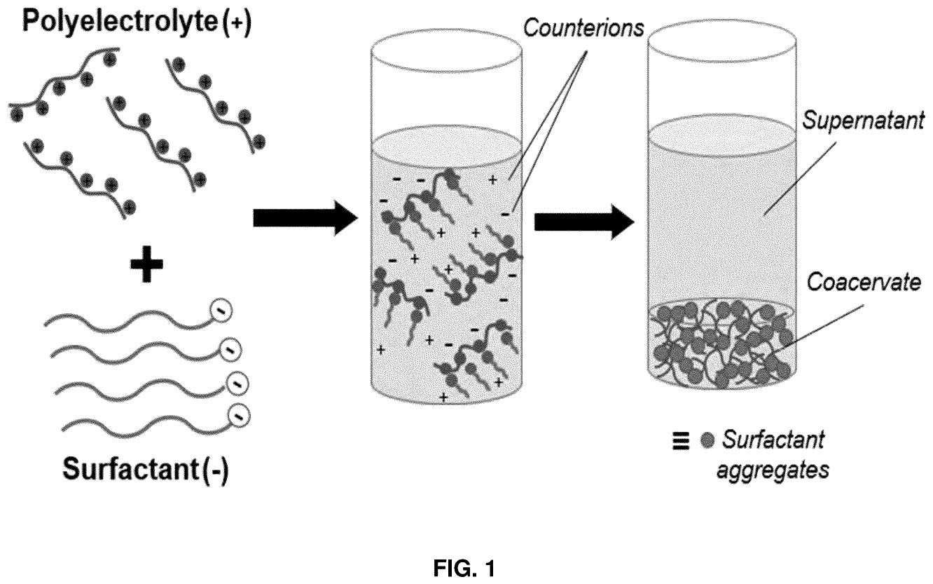

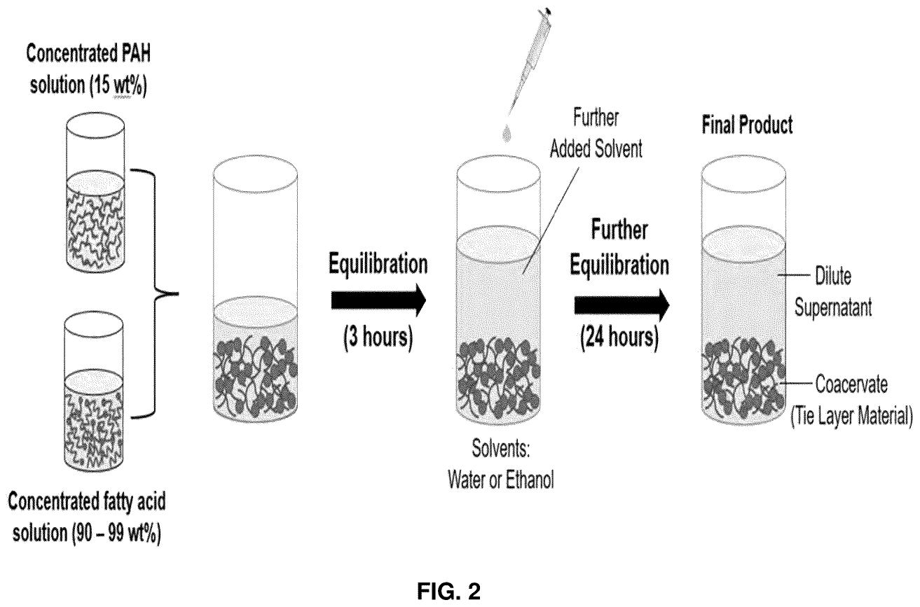

Complex coacervates can be formed through a complexation between the polymer chain units of a polyelectrolyte and an oppositely charged surfactant in a solvent. This complexation leads to an associative liquid-liquid phase separation, which forms a coacervate and a supernatant phase (FIG. 1). The coacervates are formed through spontaneous self-assembly, which may optionally be followed by further solvent addition/exchange (FIG. 2). Thus, provided herein are coacervate compositions that include a complex between a polyelectrolyte and a surfactant, and further include a solvent, which may later be removed to harden the coacervates during their use. For clarity, as used herein, the phrase “complex between a polyelectrolyte and a surfactant” includes a complex involving a polyelectrolyte ion and/or a surfactant ion. Advantageously, the formed coacervates have macroscopic dimensions and can form macroscopic films.

In certain embodiments, the coacervates described herein are moldable, adhesive, and/or viscoelastic. By “moldable” it is meant that the coacervates are capable of being shaped or molded into a specific form or structure. When a coacervate is moldable, the coacervate can be manipulated into a specific shape or structure by external forces, such as pressure or shear. By “adhesive” it is meant that the coacervates are capable of adhering to other surfaces or particles. By “viscoelastic” it is meant that the coacervates have the ability to exhibit both viscous (fluid-like) and elastic (solid-like or gel-like) behavior under different conditions.

Polyelectrolytes are polymers with ionizable repeating groups that dissociate into macroions and counterions in ionizing solvents. Polyelectrolytes are used in diverse applications. Some of their conventional applications include their use as additives to modify solution viscosity or induce gelation, as adsorbents and flocculants in water treatment, and as stabilizers for colloidal dispersions. In some embodiments, polyelectrolytes include amine groups. In some embodiments, polyelectrolytes are deprotonated polymers which are protonated, and thereby charged, upon mixture with a surfactant.

One non-limiting polyelectrolyte is polyallylamine (“PAH”), which has the following structure:

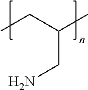

where n is an integer. However, many other polyelectrolytes are useable to make the coacervate compositions and tie layers described herein and are encompassed within the scope of the present disclosure. For example, PAH derivatives, such as quaternized PAH derivatives, as well as polyvinylamine, poly(dimethyl ammonium) salts, polyethyleneimines, and biopolymers such as chitosan or polylysine may also be used to make the coacervate compositions described herein. However, some polyelectrolyte types (e.g., some branched polyethyleneimines) may not result in the same adhesion properties. Also, chitosan is insoluble in some organic solvents, so to form an ethanolic coacervate composition with chitosan may involve mixing the ethanol with water or using ethanol-free water as the solvent. The use of chitosan, though, allows for the creation of bio-based coacervate compositions.

Surfactants are effectively used as the primary building blocks (i.e., the main components by weight other than the solvent) of the coacervate compositions. Surfactants are amphiphilic molecules that have hydrophilic heads and hydrophobic tails. Surfactants tend to adsorb at surfaces and lower the interfacial free energy, and self-assemble into colloidal structures (e.g., micelles) in aqueous solutions. Surfactants have many commercial uses including the solubilization of hydrophobic compounds, and as soaps, detergents, and wetting agents. Surfactants are generally classified according to the charge of their hydrophilic heads as being nonionic, cationic, anionic, or zwitterionic (also known as amphoteric). Cationic surfactants include cationic head groups such as primary, secondary, or tertiary amines, quaternary ammonium salts, cetyltrimethylammonium bromide, cetylpyridinium chloride, benzalkonium chloride, benzethonium chloride, dimethyldioctadecylammonium chloride, or dioctadecyldimethylammonium bromide. Anionic surfactants include anionic head groups such as sulfate, sulfonate, phosphate, or carboxylate. Zwitterionic surfactants have both cationic and anionic head groups attached to the same tail. Nonionic surfactants have covalently bonded oxygen-containing hydrophilic groups bonded to their hydrophobic tails, resulting in hydrogen bonding that makes the head hydrophilic. Example nonionic surfactants include fatty alcohol ethoxylates, alkyl glucosides, and sorbitan alkanoates.

Fatty acids, such as saturated fatty acids or unsaturated fatty acids, can serve as surfactants. As used herein, the term “fatty acid” means a carboxylic acid (or its salt) having a hydrocarbon chain and a terminal carboxyl group. The number of carbons in the hydrocarbon chain may range from 6 carbons to 30 carbons. Saturated fatty acids (i.e., fatty acids without double bonds) generate highly ordered complexes with polyelectrolytes having low solubility of the hydrophobic fatty acid tails in both water and ethanol solvents. These saturated fatty acids in complexes with polyelectrolytes generate flaky precipitates, which are not useful for adhesion or lamination. Therefore, in some embodiments, the surfactant is an unsaturated fatty acid.

Unsaturated fatty acids are fatty acids having at least one double bond in the hydrocarbon chain. Certain unsaturated fatty acids include cis double bonds in the hydrocarbon chain. A cis double bond is one in which the substituents are on the same side of a plane. One non-limiting example of an unsaturated fatty acid with at least one cis double bond is oleic acid, which has the following structure:

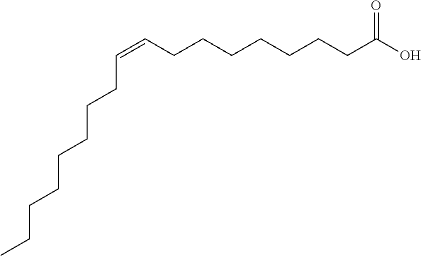 [SMILES: CCCCCCCC/C=C\CCCCCCCC(=O)O]

Another non-limiting example of an unsaturated fatty acid at least one cis double bond is linoleic acid, which has two cis double bonds and has the following structure:

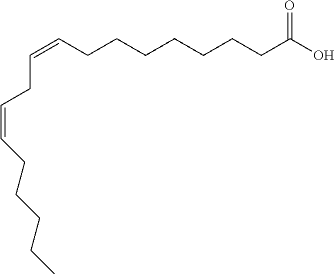 [SMILES: CCCCC/C=C\C/C=C\CCCCCCCC(=O)O]

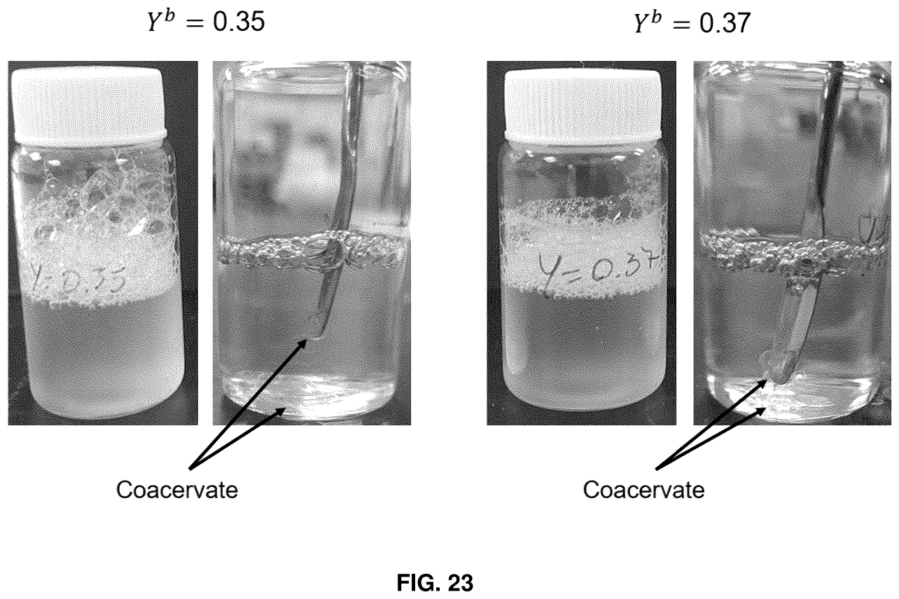

Without wishing to be bound by theory, it is believed that the cis double bond creates a kink in the tail of the surfactant, which disrupts the packing within the surfactant complex with the polyelectrolyte and, consequently, either plasticizes or liquifies the surfactant/polyelectrolyte complex. Accordingly, a surfactant having a cis double bond, including a surfactant other than a fatty acid, may be used to complex with a polyelectrolyte to form a coacervate composition. (Stearic acid, which does not have any double bonds, was used as a negative control in the examples herein.) Normally, unless both high ionic strengths and mixtures of ionic and nonionic surfactants are used, surfactants and polyelectrolyte mixtures form precipitates with solid- or gel-like properties, not tacky liquids; and, even when liquid coacervates are formed using ionic/nonionic surfactant mixtures at high ionic strengths, these coacervates lack adhesive properties (FIG. 23). However, by using a surfactant with a cis double bond, macroscopic coacervates with desirable adhesion, encapsulation, and controlled release properties can be formed. Although oleic acid and linoleic acid are described for exemplary purposes, many other surfactants, including many other unsaturated fatty acids having one or more cis double bonds, are possible in the coacervate compositions described herein and are encompassed within the scope of the present disclosure. For example, certain unsaturated fatty acids have a combination of at least one cis double bond and at least one trans double bond, and such unsaturated fatty acids are nonetheless usable in the coacervate compositions and tie layers described herein.

The complexation between a polyelectrolyte and a surfactant having a hydrophobic tail can generate adhesion or processable coatings and macroscopic solid materials useful for many applications. Furthermore, the surfactant in the coacervate composition may actually be a mix of two or more surfactants. For example, the surfactant may include a mix of one unsaturated fatty acid and one saturated fatty acid, or a mix of multiple of nonionic, cationic, and anionic surfactants. At least one surfactant in any such mix, though, should include a cis double bond to ensure formation of the coacervate complex with the polyelectrolyte.

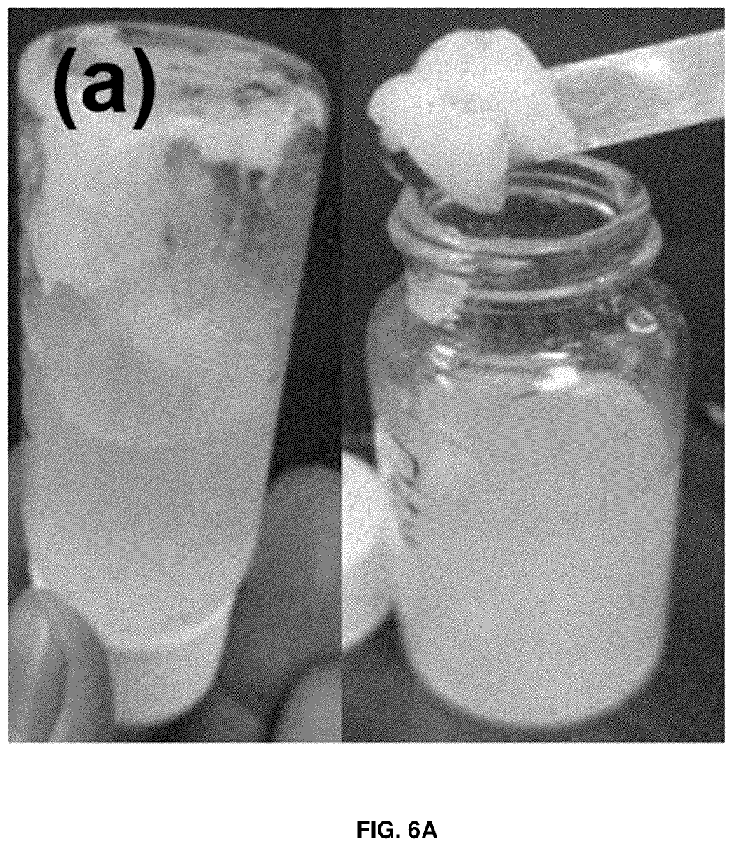

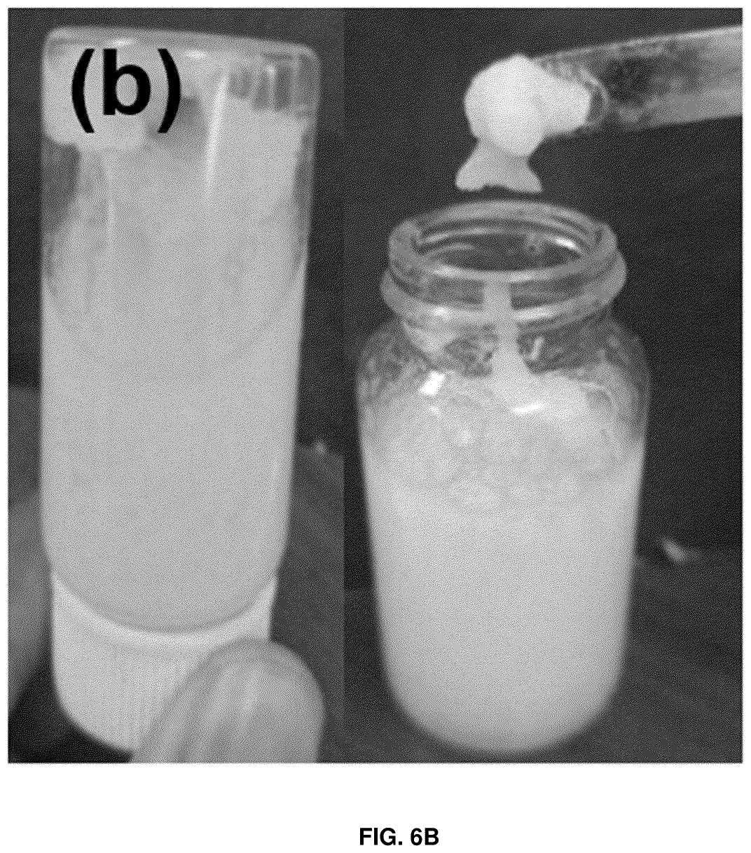

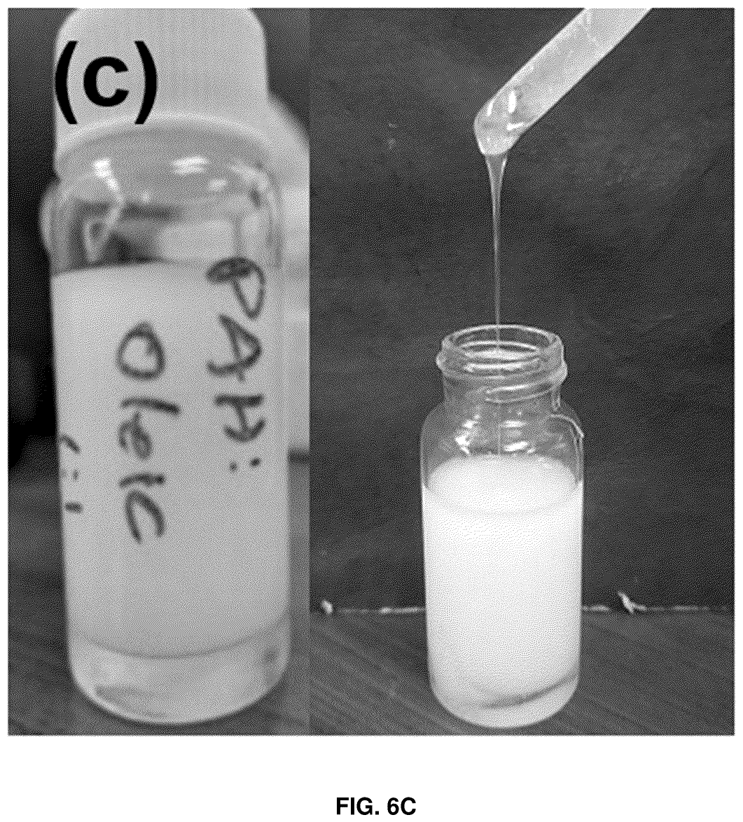

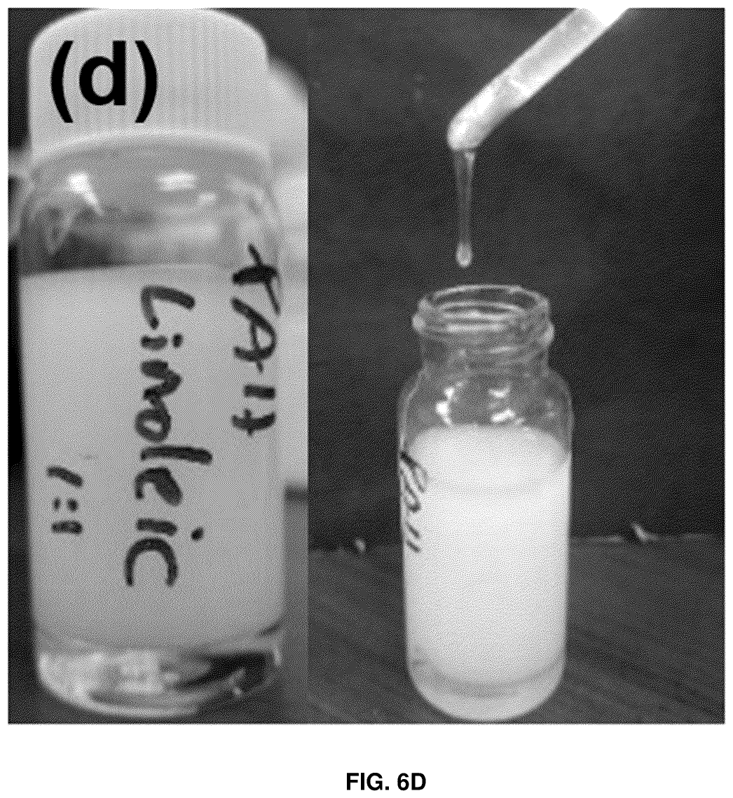

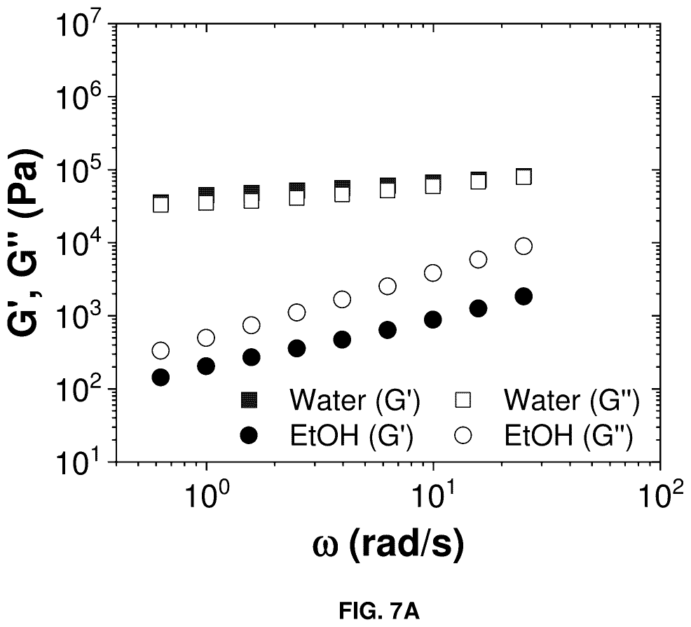

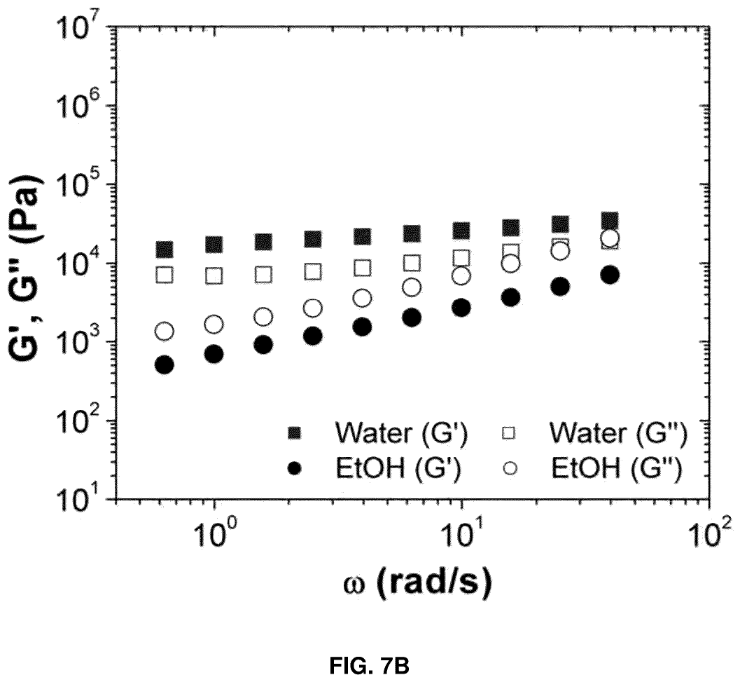

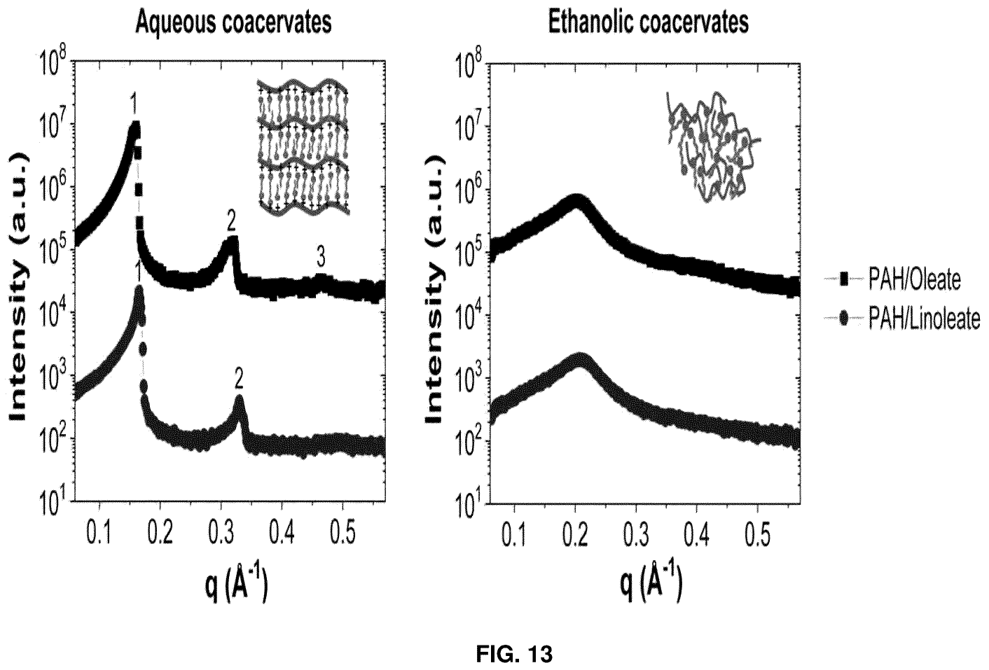

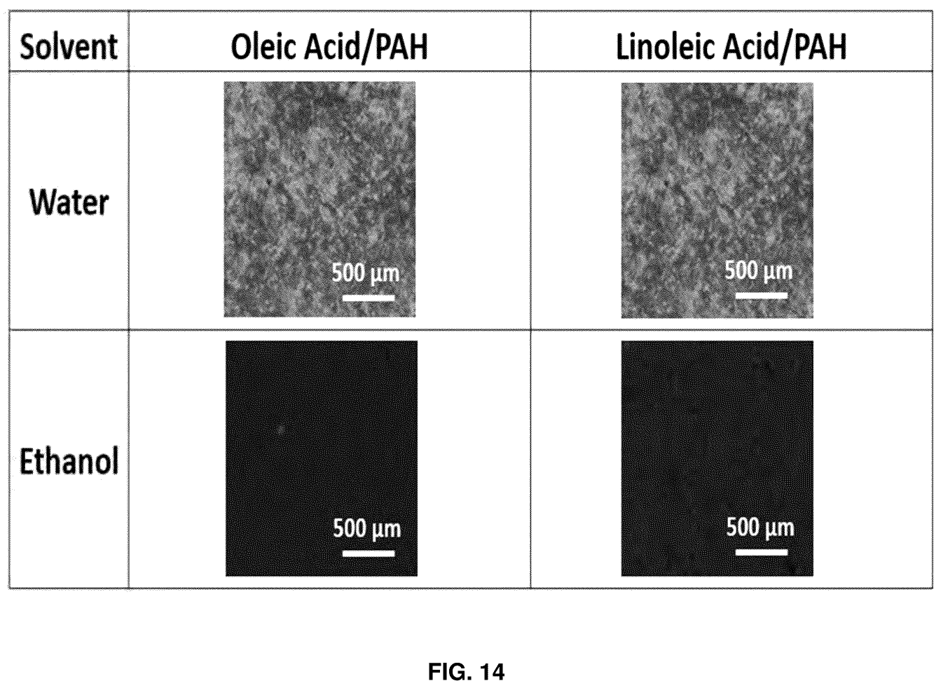

As shown in the examples herein, the choice of solvent influences the rheological properties of the coacervate composition. For example, solvation in ethanol produces viscous liquid coacervates while solvation in water generates viscoelastic solids. In water, the coacervate composition forms a putty-like solid that is sticky and will hold its shape (FIGS. 6A-6B). In ethanol, the coacervate composition becomes a viscous liquid that can flow, which makes it easier to apply (FIGS. 6C-6D). The examples herein demonstrate that the solvent selection provides access to either viscous liquid or viscoelastic solid coacervates (FIGS. 6A-6D, 7A-7B). Aqueous samples maintain a liquid crystalline structure, because water is a poor solvent for hydrocarbons. Ethanolic samples, on the other hand, have an amorphous microstructure, and the microstructural order is likely disrupted by the solvation of fatty acid tails (FIG. 13). Furthermore, aqueous samples in solution exhibit birefringence due to anisotropic, liquid crystalline ordering, whereas ethanolic samples in solution are nonbirefringent, confirming an isotropic, amorphous structure (FIG. 14). However, though water and ethanol are described for exemplary purposes, many other solvents are possible and encompassed within the scope of the present disclosure. For example, short chain alcohols other than ethanol, such as methanol or propanol, may be utilized. Alcohol solvents make it easier to dry the solvent off (i.e., evaporate the solvent), which is desired when forming films with the coacervate compositions.

Coacervate compositions can be formed using a wide range of concentrations of the polyelectrolyte and the surfactant. There is a stoichiometric relation between the carboxylate (or other ionizable group) of the surfactant and the amine (or other ionizable) group of the polyelectrolyte. The charge ratio of surfactant:polyelectrolyte may range from about 0.05:1 to about 20:1, or from about 0.25:1 to about 5:1. The charge ratio can be, for example, 1:1 surfactant:polyelectrolyte, or 2:1 surfactant:polyelectrolyte. Advantageously, this allows for the coacervate composition to be mostly biobased in some embodiments, such as by being over 80% by dry weight fatty acid (i.e., over 80% by weight excluding the solvent). Advantageously, the surfactant/polyelectrolyte coacervates can form from common ingredients (including biobased surfactants and polymers), and do not require special conditions to maintain their processable properties (i.e., their formation does not rely on high ionic strengths or the presence of specific amounts of additional/nonionic surfactant species).

In some embodiments, the polyelectrolyte is PAH, the surfactant is an unsaturated fatty acid having one or more cis double bonds (such as oleic acid or linoleic acid), and the solvent is either water or ethanol.

One non-limiting example coacervate composition includes an oleic acid and PAH complex in water. Another non-limiting example coacervate composition includes an oleic acid and PAH complex in ethanol. Another non-limiting example coacervate composition includes a linoleic acid and PAH complex in water. Another non-limiting example coacervate composition includes a linoleic acid and PAH complex in ethanol.

In some embodiments, the surfactant includes one or more substituents in the carbon chain. Non-limiting example substituents include halogens, alkyl groups, hydroxyl groups, and carboxyl groups. The substituent(s) may serve many possible purposes, such as enhancing the solubility of the surfactant in a desired solvent.

The coacervate composition may further include one or more additives. Suitable additives include, but are not limited to, dyes, disinfectants, drugs, fragrances, flavors, any actives in a household application, other surfactants, oils, and solid particles with dimensions smaller than those of the film (e.g., particles from about 10 nm to about 1 mm in diameter). Provided that the additive does not prevent complexation between the polyelectrolyte and the surfactant, and is soluble or dispersible in the solvent, there is no limit on the type of additive that may be incorporated into the coacervate composition.

The coacervate composition may be sticky and therefore useful as an adhesive. The coacervate compositions may be sticky even before adding the extra solvent or after allowing some of the solvent to evaporate. Furthermore, the coacervate composition may be transparent, and therefore useful in transparent packaging or other plastic products.

As demonstrated in the examples herein, the coacervate composition may be utilized to controllably release a payload. The payload may be any solute, such as a drug, a fragrance, a dye, or any other small molecule that can be kinetically trapped by the coacervate composition. In this manner, the coacervate composition may be used in applications such as for drug delivery, or as an air freshener with the sustained release of a fragrance. Any modestly hydrophobic molecule partitions well into the coacervate composition. Rhodamine B (RhB), the dye used in the examples herein, is not particularly hydrophobic, but was still taken up by the coacervate composition, giving a bright pink coacervate and a clear liquid supernatant. However, the payload should not be a species which does disrupt the surfactant/polyelectrolyte complex, such as a concentrated multivalent alkaline or transition metal ion salt (e.g., concentrated CaCl₂)) or a polyvalent molecule which could form coacervate-destroying competitive complexes.

As further demonstrated in the examples herein, the coacervate composition may have oxygen scavenging properties. Oxygen scavenging properties are especially useful in food packaging applications, to aid in preserving packaged food items for longer periods of time. Without wishing to be bound by theory, it is believed that the oxygen scavenging properties of the coacervate compositions come from the double bond(s) in the surfactant. The allylic C—H groups in such surfactants may be oxidized in the presence of oxygen. Linoleic acid in particular has two double bonds and is autocatalytic. Oleic acid, however, is not autocatalytic, and therefore may need the presence of a catalyst, such as cobalt acetate, to effectively scavenge oxygen. Accordingly, in some embodiments, the coacervate composition includes oleic acid as the surfactant and further includes cobalt acetate.

A high pH solution dissolves the coacervate compositions because the high pH effectively turns off the charge that leads to the complexation forming the coacervates. The exact pH at which the coacervate compositions dissolve depends on the specific composition, but a solution with a room-temperature pH of about 12 should dissolve any of the coacervate compositions described herein. However, this property can be tweaked by additives present in the coacervate composition or changing the molecular structure of the polyelectrolyte or surfactant. An elevated temperature also helps the dissolution process. For example, 100° C. works very well to dissolve the coacervate compositions in a 1 M NaOH solution (which has a room-temperature pH of 14), with 1×10³-1×10² M NaOH (whose room-temperature pH is 11-12) still dissolving the coacervate (albeit much more slowly) at this temperature. Further temperature elevation may allow for a still slightly lower pH solution to be used to dissolve the coacervate composition. At room temperature, there may be some reprecipitation of the surfactant; however, nonionic surfactants can solubilize fatty acids to avoid this. Therefore, if a nonionic surfactant is present in the coacervate composition, reprecipitation at room temperature may be reduced or altogether avoided.

The coacervate composition can be coated to create a film on a support. The coacervate composition can be deposited in a liquid form as a dispersion and can leave behind a uniform solid coating. Such dispersion-based application facilitates uniform spreading of the otherwise viscous coacervates and enables them to be coated more homogeneously. To make a coating, the coacervate composition may be spread onto a substrate and then dried at 25° C. for a period of time, such as for about 40 minutes or an hour, until the desired coating is achieved. As shown in the examples herein, the deposition of coacervate compositions can be coated more homogeneously if they include a solvent-soluble electrolyte so as to induce coalescence and aid in the formation of a uniform coating or film. Suitable electrolytes include, but are not limited to, NaNO₃ or NaCl, or other alkali metal salts, halide salts, other nitrates, ammonium salts, sulfates, or phosphates. Any salt soluble in the solvent can be used to induce coalescence of the colloidal coacervate dispersion. There is also no limit to the type of support the coacervate composition can be coated on. Non-limiting example supports include plastic materials such as PET, LDPE, EVOH, nylon, or PEVA, as well as non-plastic materials such as glass, wood, fiberglass, fabrics, and metals.

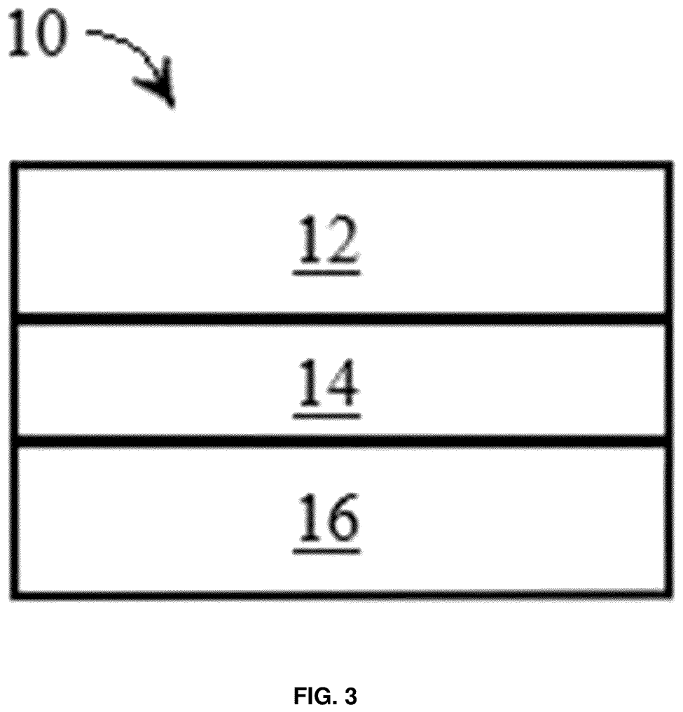

A coating or film of the coacervate composition can be utilized in a multilayer packaging material. Referring now to FIG. 3, a first embodiment of a multilayer packaging material 10 is depicted. The multilayer packaging material includes a first plastic layer 12, a tie layer 14, and a second plastic layer 16 laminated together. The tie layer 14 acts to join the first plastic layer 12 together with the second plastic layer 16. The tie layer 14 is a film of a coacervate composition as described herein. The first plastic layer 12 and the second plastic layer 16 are each independently a plastic material such as, but not limited to, PET, LDPE, EVOH, nylon, or PEVA.

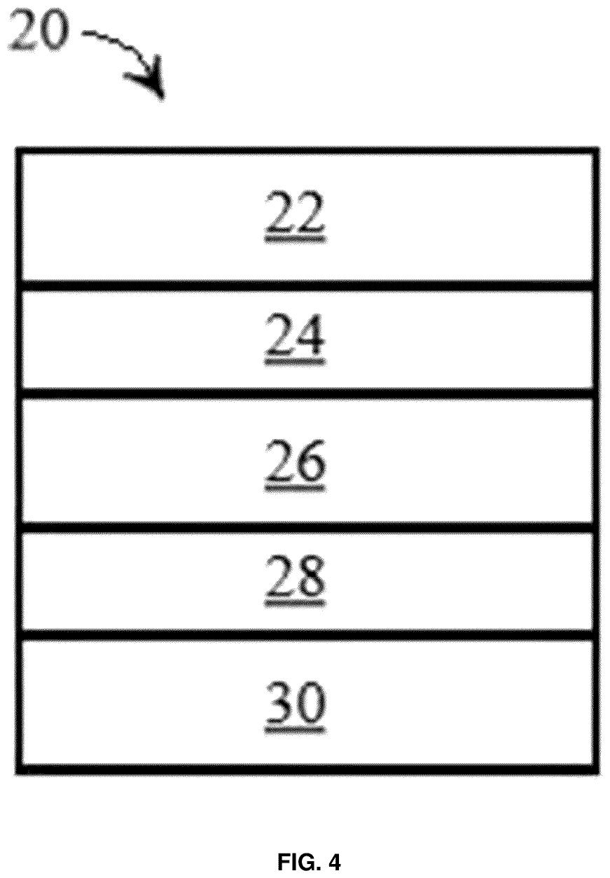

Referring now to FIG. 4, a second embodiment of a multilayer packaging material 20 is depicted. The multilayer packaging material 20 includes a first plastic layer 22, a first tie layer 24, a second plastic layer 26, a second tie layer 28, and a third plastic layer 30 laminated together. The first tie layer 24 acts to join the first plastic layer 22 together with the second plastic layer 26, and the second tie layer 28 acts to join the second plastic layer 26 together with the third plastic layer 30. One or both of the first tie layer 24 and the second tie layer 28 is a film of a coacervate composition as described herein. The first plastic layer 24, the second plastic layer 26, and the third plastic layer 30 are each independently a plastic material such as, but not limited to, PET, EVOH, LDPE, nylon, or PEVA.

It is understood that although FIGS. 3-4 depict, for exemplary purposes, multilayer packaging materials having three layers and five layers, respectively, there is no limit to the number of layers in a multilayer packaging material in accordance with the present disclosure. For example, a multilayer packaging material may include fifteen layers, where any one or more layers is a tie layer comprising a coacervate composition described herein. In other examples, a multilayer packaging material may include several hundred layers, where any one or more layer is a tie layer comprising a coacervate composition described herein.

In some non-limiting examples, a multilayer packaging material includes a first plastic layer of PET, a tie layer of a coacervate composition made from PAH, oleic acid or linoleic acid, and a water or ethanol solvent, and a second plastic layer of LDPE or HDPE.

Referring again to FIGS. 3-4, the multilayer packaging materials 10, 20 may be produced by simply laminating the coacervate compositions together with the plastic materials. Advantageously, the coacervate compositions may be laminated together with dissimilar plastic materials.

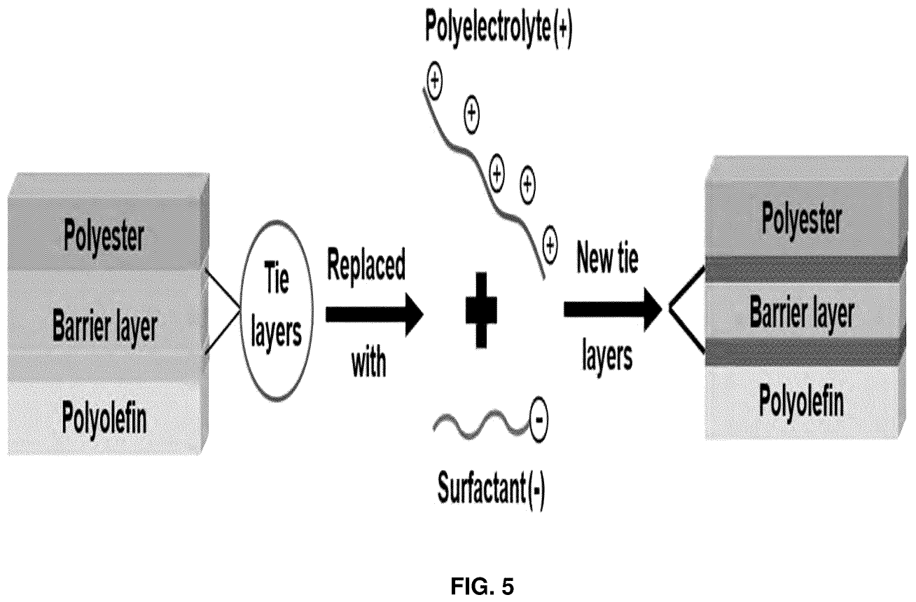

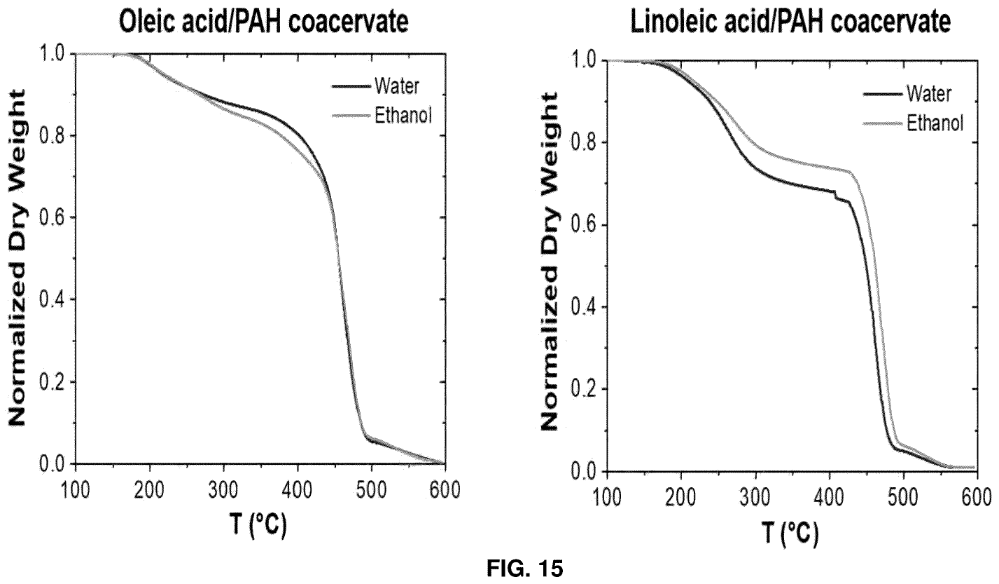

Referring still to FIGS. 3-4, the tie layers 14, 24, 28 can be completely dissolved in a basic solution, which effectively removes the charge holding together the complexes. For example, in 1 M NaOH at 100° C., the tie layers can be completely dissolved. This tie layer dissolution allows for the delamination and separation of the different layers by the techniques used in conventional recycling methods (e.g., density-based separation) to then occur. As demonstrated in the examples herein, tie layers made from the coacervate compositions are thermally stable up to about 150-190° C. (FIG. 15). Thus, the tie layers dissolve on demand under normal recycling process conditions and can replace conventional tie layers in multilayer packaging materials (FIG. 5). Advantageously, this on-demand dissociation can be combined with the oxygen scavenging properties described herein to produce tie layers that also reduce oxygen permeation.

The compositions described herein may also be made available via a kit containing one or more key components. A non-limiting example of such a kit comprises an ionizable polymer (that can serve as a polyelectrolyte) and a surfactant having a cis double bond in separate containers, where the containers may or may not be present in a combined configuration. Many other kits are possible, such as kits that further include a solvent such as water or ethanol. In other embodiments, a kit for making a coacervate composition may include a first container housing a polyelectrolyte, and a second container housing a mixture of a solvent and a surfactant having a cis double bond and a hydrophobic tail. In other embodiments, a kit for making a coacervate composition may include a first container housing a surfactant having a cis double bond and a hydrophobic tail, and a second container housing a mixture of a solvent and a polyelectrolyte. In other embodiments, a kit for making a coacervate composition may include a first container housing a first mixture of a solvent and a polyelectrolyte; and a second container housing a second mixture of the solvent and a surfactant having a cis double bond and a hydrophobic tail.

The kits typically further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### Examples

### Coacervate Formation and Rheology

Upon the mixing of aqueous 15 wt % PAH solution with neat fatty acids, the protons on the fatty acid molecules were transferred to the PAH amine groups, thus resulting in ionically associated cationic amine groups and anionic carboxylate groups (which formed upon the proton transfer from the fatty acids to the PAH amine groups). This complexation, and the hydrophobic association of the polymer-bound aliphatic fatty acid tails, produced complex coacervates. When the complexes were placed in water, they were putty-like (i.e., they were moldable but held their shape under gravity over multiple months; FIGS. 6A, 6B). Conversely, when these complexes were solvated in ethanol, they became much more fluid-like (FIGS. 6C, 6D). This shift from a viscoelastic solid to a liquid behavior, which was also characterized by dynamic rheology (FIGS. 7A, 7B), corresponded to a significant drop in both dynamic moduli (as well as in the storage modulus, G′, becoming largely lower than the loss modulus, G″) and made the complexes much easier to spread on a surface. Regardless of whether these coacervates were aqueous or ethanolic, however, they were tacky (indicating their ability as adhesives) and spreadable/processable.

Conversely, PAH/stearate complexes (i.e., which lacked the plasticizing double bonds) formed solid-like precipitates, which were not moldable and were difficult to spread (regardless of whether they were solvated in water or ethanol). Thus, the introduction of double bonds into the surfactant tails (e.g., the use of unsaturated fatty acids) enables the generation of processable coacervates from a single, oppositely charged surfactant/polyelectrolyte pair. This approach may greatly expand the range of soft material/complex fluid properties that can be accessed through surfactant/polyelectrolyte complexation (as well as the range of conditions/compositions under which such coacervates can be formulated). This effect of fatty acid unsaturation likely reflects less ordered packing within the resulting fatty acid/polyelectrolyte complex, which hinders the hydrophobic association between the aliphatic tails of the polymer-bound fatty acid molecules and produces more fluid-like rheological behavior. Indeed, when the degree of saturation is increased from one double-bond (for oleic acid) to two double bonds (for linoleic acid), this effect is (at least in the aqueous coacervates) magnified, as seen by the lower dynamic moduli of the PAH/linoleate complexes (cf. FIGS. 7A and 7B). Moldable coacervates also form upon the aqueous mixing of oleic acid with branched polyethyleneimine (BPEI) and chitosan. Thus, processable fatty acid-based coacervates can be generated from a variety of (both synthetic and biobased) polycations, and further findings described below indicate these coacervates to have many diverse applications.

### Controlled Deposition of Coacervate Films

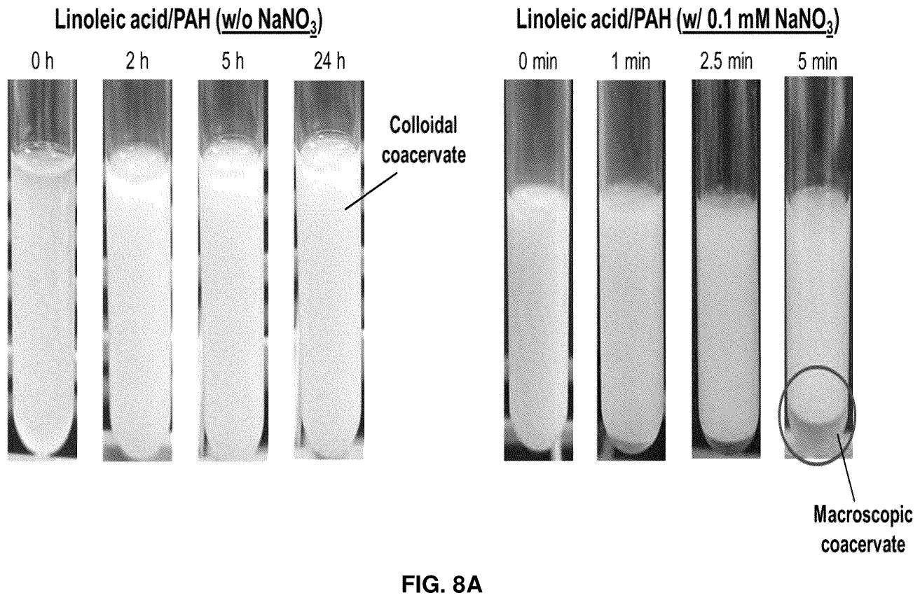

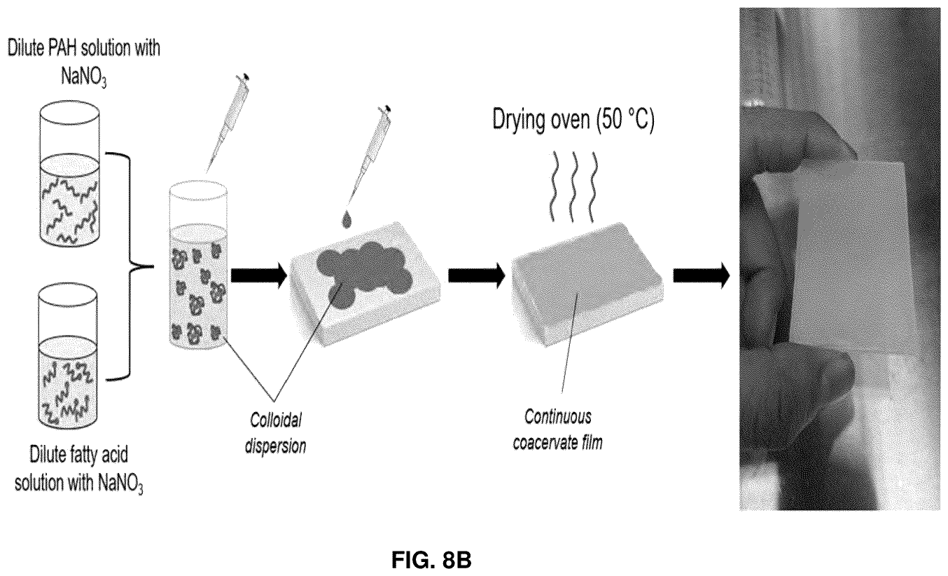

Besides beginning to characterize their bulk properties, it has been observed that it is possible to apply these coacervates as films. While doing so by spreading bulk coacervates is complicated by their high viscosity and adhesion to the tools used to spread them, these challenges can be overcome by preparing colloidally dispersed coacervate droplets (which readily form when the surfactants and polyelectrolytes are diluted) and coalescing/depositing these droplets onto surfaces. To this end, titrating 20 wt % ethanolic oleic or linoleic acid solutions into 2 wt % ethanolic PAH solutions generated low-viscosity dispersions of submicron coacervate droplets. Without added electrolyte, these coacervates were colloidally stable and did not coalesce into films. When a small amount of ethanol-soluble screening electrolyte (e.g., 0.1 mM NaNO₃) was added, however, they coalesced into macroscopic coacervate phases at rates that could be tailored by varying either the electrolyte concentration or the fatty acid: PAH ratio (FIG. 8A). When these destabilized low-viscosity dispersions were spread onto a surface and allowed to partially dry, they coalesced into smooth, continuous, and adhesive coacervate films (FIG. 8B). The thickness of the surfactant/polyelectrolyte coacervate films can be tailored according to the desired application parameters.

### Performance as a Stimulus-Responsive Adhesive

PAH/oleate and PAH/linoleate coacervates (such as the film shown in FIG. 8B) readily adhere to both high- and low-energy surfaces. The former was evident from their adhesion to both glass vials and metallic spatulas, while the latter was quantified by allowing these coacervates to (in their dried states) serve as tie layers for the adhesion of LDPE to PET. Upon sandwiching these coacervates between LDPE and PET sheets, they (upon solvent removal) produced significant adhesion, with lap shear bond strengths of ≥100 kPa. (When LDPE was used, the strong adhesion prompted the coacervate to be initially applied to the LDPE and then covered with PET, and was not so strong when the coacervate was first applied to PET. In the case of the PET/EVOH adhesion, on the other hand, strong adhesion could be achieved independent of the addition order. Accordingly, it may be beneficial to apply the coacervate to the more hydrophobic substrate first.) This coacervate application is assisted by their solvation in ethanol. Not only does ethanol facilitate the spreading of the fatty acid/polyelectrolyte complexes on the adhesion substrate (by serving as another disruptor of hydrophobic interactions between the polyelectrolyte-bound fatty acid molecules), but, being more volatile than water, it facilitates the drying of the adhesive tie layer upon its application (and, thus, promotes the rapid achievement of significant adhesion).

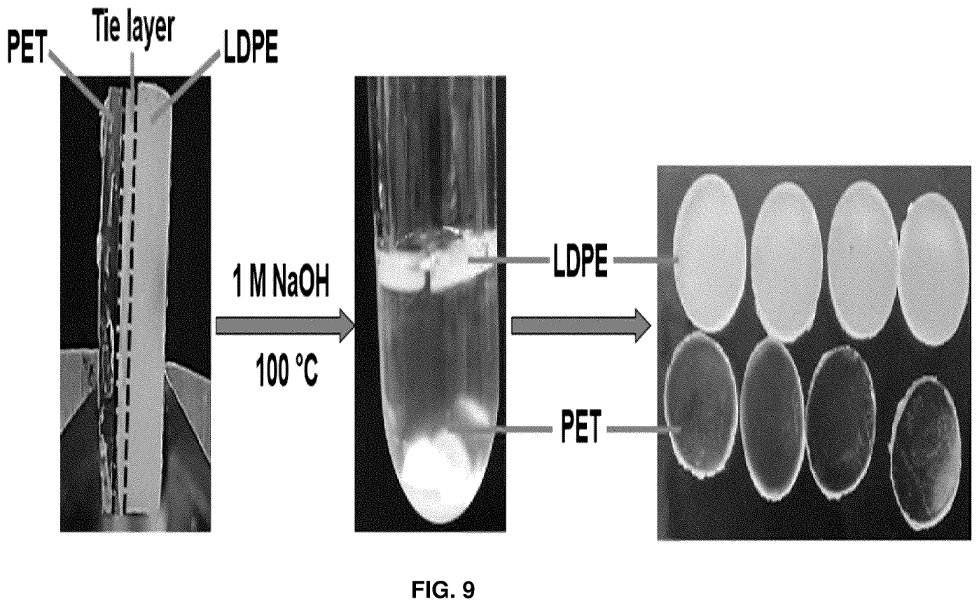

A key property of these fatty acid/polyelectrolyte coacervates, when used as adhesives/tic layers for multilayer plastic films, is their ability to dissociate on demand under conditions present in conventional chemical recycling processes (i.e., in 1 M NaOH). This dissociation reflects: (1) the reversibility of surfactant/polyelectrolyte association; (2) the weakening of this electrostatic association at elevated ionic strengths; and (3) the deprotonation of the PAH amine groups at high pH, which eliminates the polyelectrolyte charge. This effect was demonstrated by using PAH/oleate and PAH/linoleate complexes to create multilayer films (FIG. 9), where they served as tie layers between PET (the clear layer) and LDPE (the opaque layer). Upon being exposed to 1 M NaOH at 100° C., these complexes dissolved, causing the multilayer films to separate (see FIG. 9, where, upon the PAH/fatty acid complex dissolution, the LDPE layer floated while the PET sunk). This type of separation is important for the recycling of multilayer films composed of disparate materials and occurred within ˜30 min for both complex types. Taken together, the above experiments indicate that cationic polyelectrolyte complexes with unsaturated fatty acids can reduce plastic waste by enabling the recycling of multilayer plastic films, whose recycling has hitherto been largely inhibited by difficulty in separating the dissimilar layers into separate recycling streams. Simultaneously, these films could reduce spoilage of foods and other perishable products, which remains another substantial source of waste (and added cost) to both manufacturers and consumers.

### Solute Uptake/Release and Barrier Properties

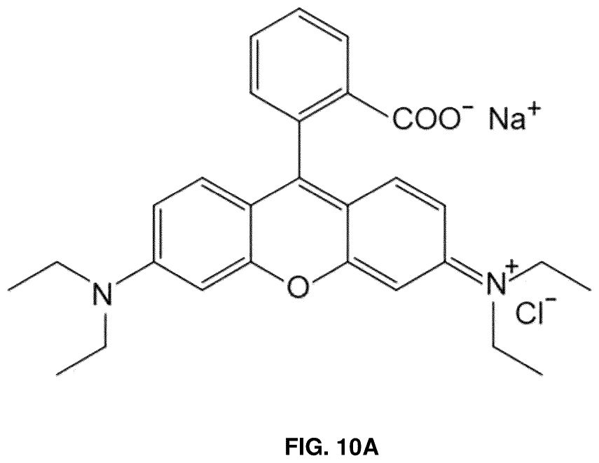

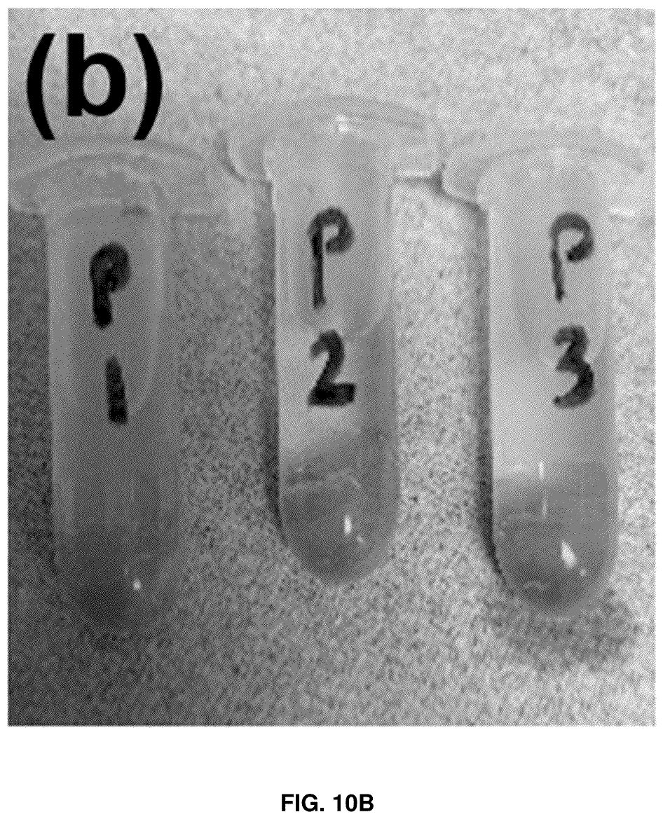

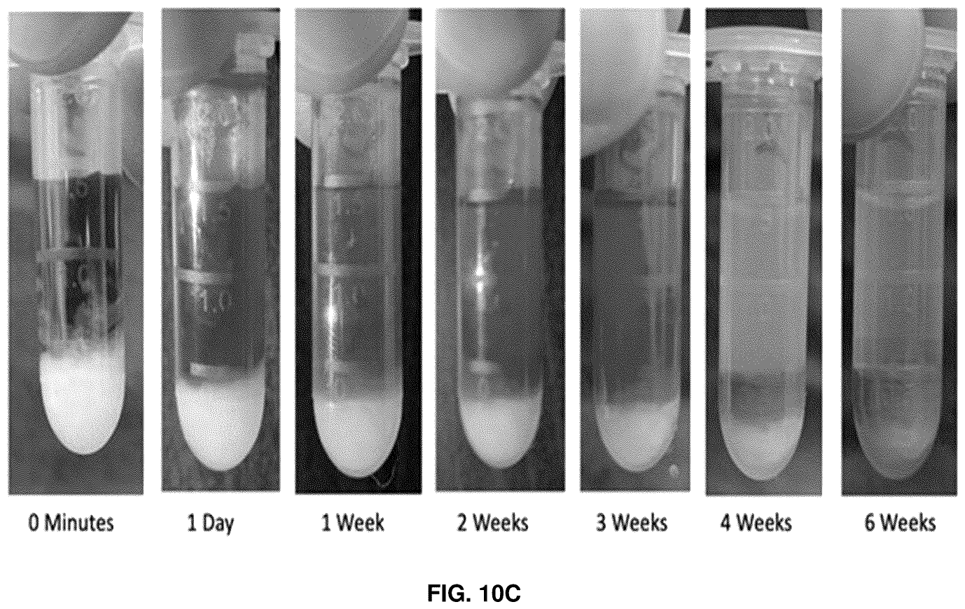

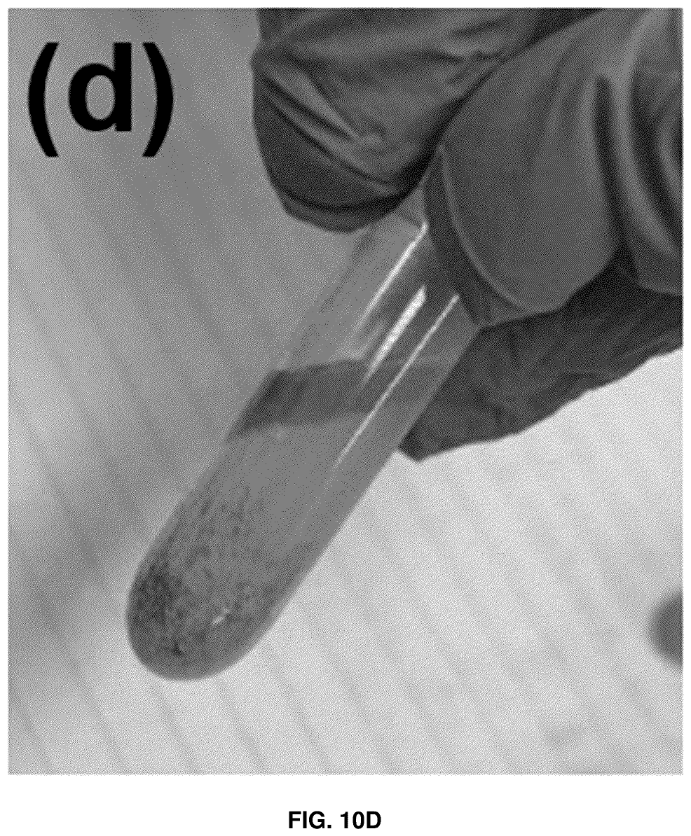

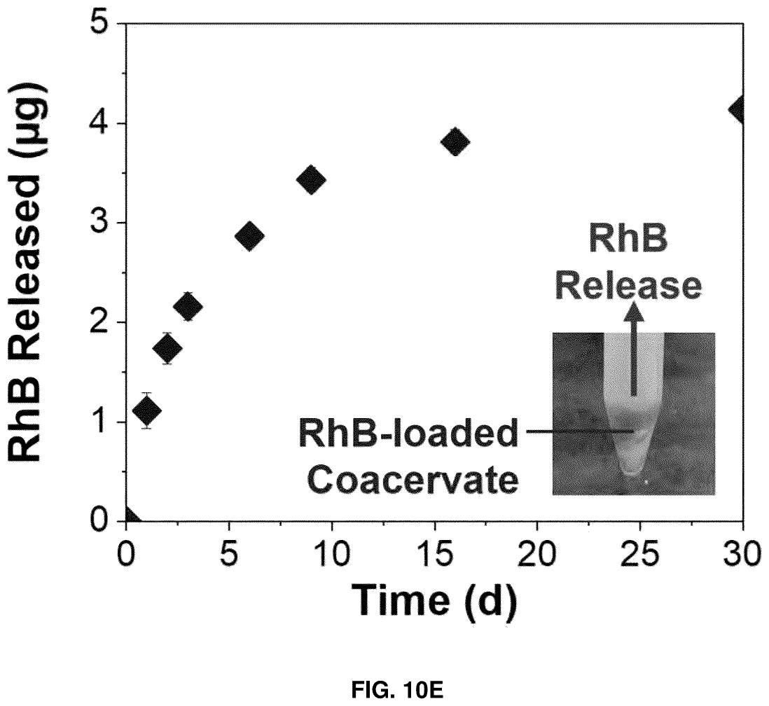

Observations on encapsulation and release properties of the coacervates indicate that they can efficiently take up small-molecule payloads and serve as effective barriers to their diffusion. Analysis of water-soluble dye RhB (FIG. 10A) uptake into PAH/oleic acid coacervates indicates that solutes (even those with moderate hydrophobicity) can strongly preferentially partition into these coacervates and that the diffusion of small-molecule solutes through these coacervates is quite slow. The preferential partitioning is evident in FIG. 10B, where nearly all the RhB partitions into the coacervate phase, while the slow diffusion is illustrated in FIG. 10C, where 0.5 mg/mL aqueous RhB solution is poured on top of the coacervate, which is adhered to the bottom of the tube. The red RhB front slowly penetrates downwards into the coacervate pellet and fails to reach the bottom of the ˜1-cm-thick pellet even after 6 weeks. Based on the scaling relationship between the characteristic time of one-dimensional diffusion (t˜L²/(2D_{A})) and the diffusion distance (estimated as the RhB penetration depth), the diffusivity of RhB within the PAH/oleate coacervate is ˜10⁸-10⁷ cm²/s, or nearly two orders of magnitude slower than it is in water or a typical water-rich hydrogel. This low diffusivity indicates that these coacervates can serve as efficient barriers for diffusion and enable the sustained release of active payloads over multiple days (or even weeks). The coacervate also exhibits minimal changes in volume throughout these experiments, which can extend the applications of these materials to situations where changes in the sustained release device dimensions (which often occur over time with hydrogels) can produce harmful effects.

Another remarkable property of these coacervates is that, unlike conventional coacervates, which are denser than water and sediment to the bottom phase of the phase-separated mixture, the polyelectrolyte/unsaturated fatty acid coacervates have a lower density than water and, when dislodged from the bottom of the tube, float. This became evident upon the centrifuging of PAH/oleate coacervates in the presence of their supernatant phases (FIG. 10D). When the freshly formed, aqueous RhB-loaded coacervates (submerged in their supernatant phases) were centrifuged, they detached from the bottoms of the test tubes and generated macroscopic, polymer-rich layers at the air-liquid interfaces. Thus, centrifuging these coacervates into pellets at the bottoms of the tubes (as shown in FIGS. 10B, 10C) required the coacervates to be separated from the dilute, supernatant phases before being centrifuged. Without wishing to be bound by theory, it is believed that this unusually low coacervate density may reflect their high hydrocarbon content. In any event, this surprisingly low density allows these multifunctional materials to be used in additional applications in the engineering of air-water interfaces. The coacervates are capable of slowly releasing a solute payload. FIG. 10C show photographs at time points of up to 6 weeks after uptake of the Rhodamine B dye. FIG. 10E shows the release of the Rhodamine B dye over time. This slow, multi-week solute release indicates that the coacervates may be utilized for applications such as household disinfection, flavors and fragrance encapsulation, and drug delivery. Preferential solute partitioning may also be useful in chemical separations.

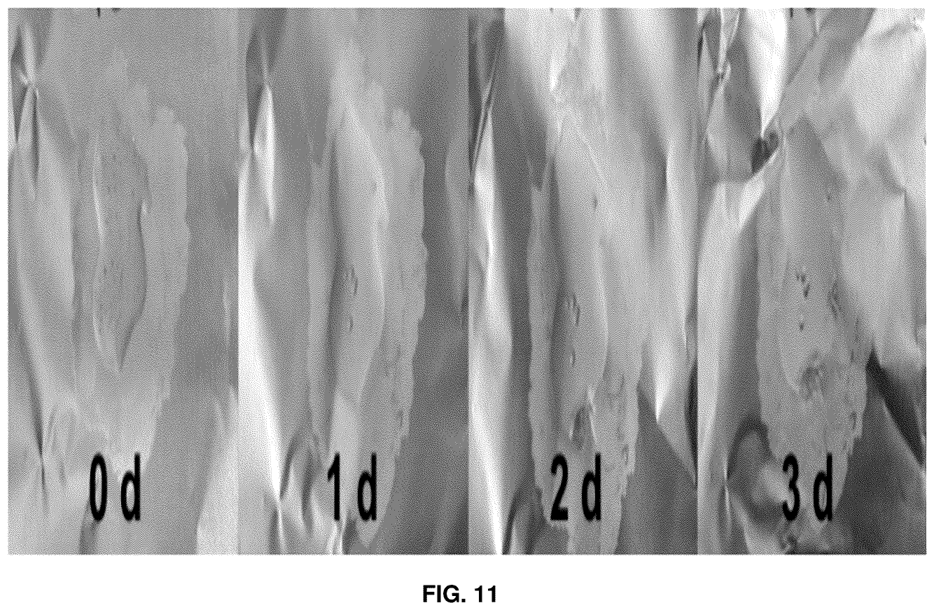

Besides their utility as diffusion barriers, coacervates prepared from fatty acids with multiple double bonds (e.g., linoleic or linolenic acid) can react with oxygen. This reactivity allows for the preparation of active barrier films, which reduce shelf-life-reducing oxygen permeation by consuming the oxygen through a chemical reaction. The incidence of this chemical reaction within the PAH/linoleate complexes is evident from the yellowing of the PAH/linoleate coacervates (see FIG. 11), as the reaction of unsaturated fatty acids with oxygen is known to produce this yellowing effect. Consequently, besides serving as adhesives and barriers to diffusion, these materials can be formulated to scavenge oxygen. Collectively, the above findings indicate that, besides their processability, these coacervates have diverse possible applications, including serving as adhesive tie layers, as active barriers used to increase the shelf-life of consumer products, and as sustained release vehicles for active compounds.

### Dissociation Kinetics

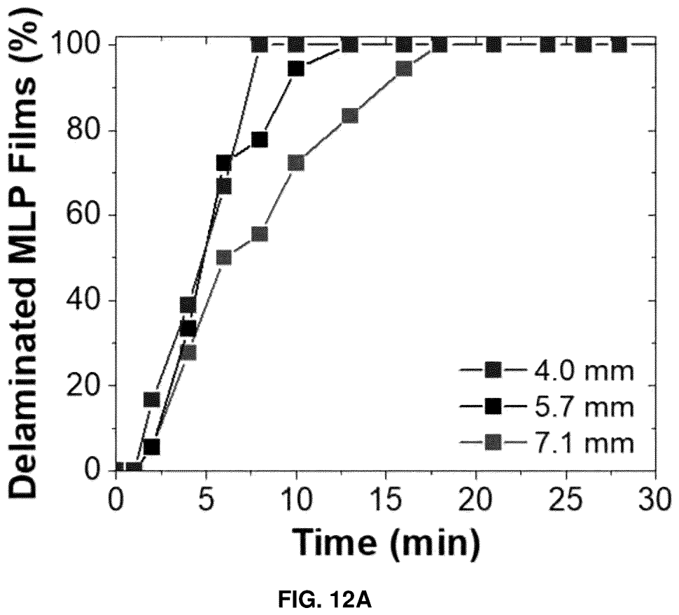

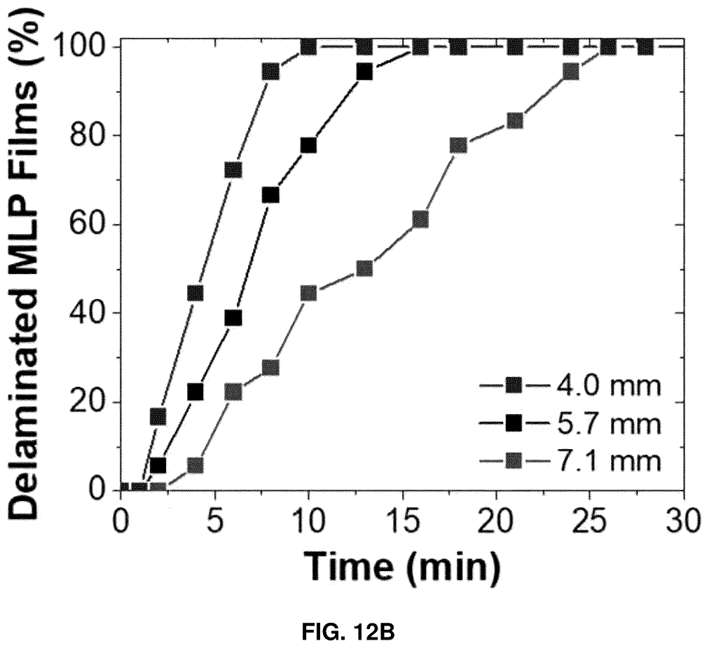

FIGS. 12A-12B show the dissociation kinetics of PET/LDPE bilayer film fragments (squares with either 4.0, 5.7, or 7.1 mm dimensions) tied together with PAH/oleate (FIG. 12A) or PAH/linoleate (FIG. 12B) complexes in 1 M NaOH at 100° C. The plots show the percent of delaminated multilayer film fragments as a function of processing time, and both PAH/fatty acid complexes were prepared at a 1:1 fatty acid: PAH monomer molar ratio. Each of these film types fully delaminated within >30 min of treatment.

### Small-Angle X-Ray Scattering (SAXS)

FIG. 13 shows that the liquid properties of ethanolic coacervates coincide with a transition to a less-ordered microstructure. Aqueous samples maintained liquid crystalline structure, as water is a poor solvent for hydrocarbons. In contrast, ethanolic samples had amorphous microstructure with the microstructural order likely having been disrupted by the solvation of the fatty acid tails.

Using polarized optical microscopy, bright patterns only form when polarized light is rotated by anisotropic samples. FIG. 14 shows polarized optical microscopy results revealing that aqueous coacervate samples in solution were birefringent due to anisotropic, liquid crystalline ordering, whereas ethanolic coacervate samples in solution were nonbirefringent, confirming an isotropic, amorphous structure.

### Thermal Stability

The thermal stability of the tie layers extends far beyond their processing and application temperatures. FIG. 15 shows the dry weight remains constant up to 150-190° C. Degradation is relatively insensitive to the solvent used during preparation.

### Performance as Tie Layer

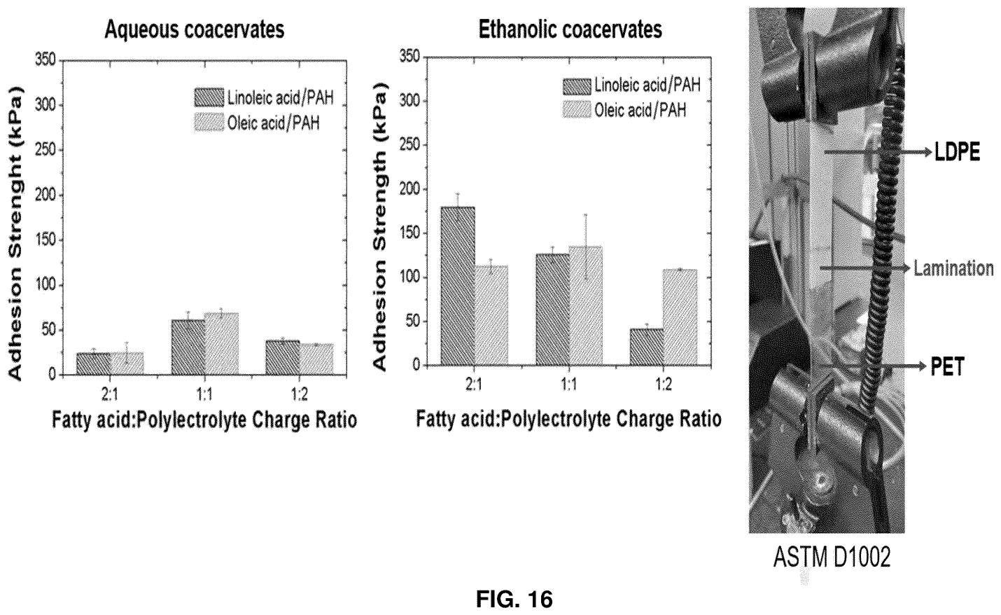

FIG. 16 shows the performance of a tie layer in an LDPE-to-PET adhesion application with (even when the tie layer is applied through uncontrolled manual spreading of viscous macroscopic coacervates with a spatula) lap shear bond strengths above 100 kPa. The multilayer composition was PET/tie layer/LDPE. Ethanol was more volatile than water, and therefore easier to remove. More thorough drying (and better surface spreading) of the ethanolic coacervates resulted in stronger adhesion.

### Coating

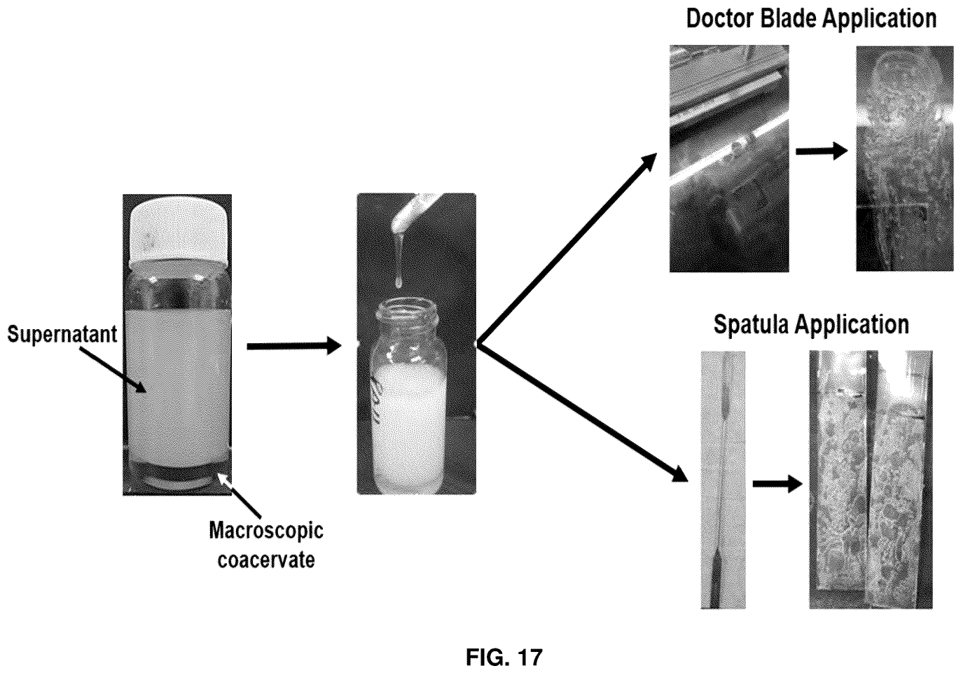

A uniform coating of preformed macroscopic coacervates is challenging to achieve. Application by either a doctor blade technique or a spatula can result in an inconsistent thickness with air bubbles, because of the difficulty in spreading the adhesive evenly (FIG. 17). However, a more uniform coating can be achieved through a colloidal deposition of the coacervate. A colloidal dispersion involves small particles (which can be solids, liquid droplets, or gas bubbles) distributed in a continuous phase (e.g., a solvent), with a particle size ranging from about 1 to 1,000 nm. The dispersions can be stable (remain dispersed) or unstable (coagulate). Colloidal coacervate dispersions can be prepared using lower PAH and fatty acid concentrations. Stable colloidal dispersions are made with a PAH concentration ranging from 0.1 wt % to 3.0 wt %, a fatty acid concentration ranging from 1 wt % to 30 wt %, and a stoichiometric ratio of 1:1. (The coacervate that forms from these mixtures tends to be significantly more concentrated. When solvated, the fatty acid content in the coacervate composition can reach about 60 wt % and can exceed 80 wt % when dried. The PAH concentration, on the other hand, can reach about 10-15 wt %.)

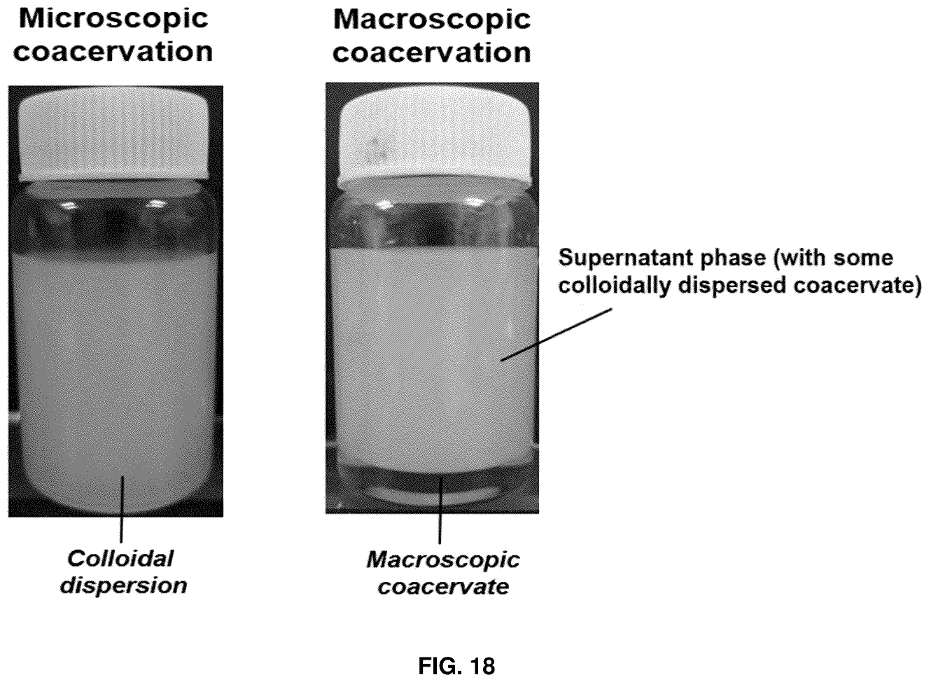

Macroscopic complex coacervation does not readily occur due to the stability of colloidal coacervate dispersions. To coagulate the colloidal dispersions into surface-deposited adhesive films, a small concentration of an ethanol-soluble electrolyte, such as NaNO₃, may be added. FIG. 18 shows photographs of the difference between a microscopic coacervation and a macroscopic coacervation. Though the turbidity of the supernatant phase in the image on the right in FIG. 18 reveals the persistence of some colloidally dispersed coacervate, nearly all the PAH and fatty acid in this mixture is sequestered within the translucent macroscopic coacervate phase at the bottom.

NaNO₃ served as an ethanol-soluble electrolyte that reduces the colloidal stability by screening electrostatic repulsion. FIG. 8A shows photographs of coacervates without NaNO₃ and coacervates with 0.1 mM NaNO₃. The colloidal coacervate droplets can be coalesced into macroscopic coacervate phases by adding 0.1 mM NaNO₃.

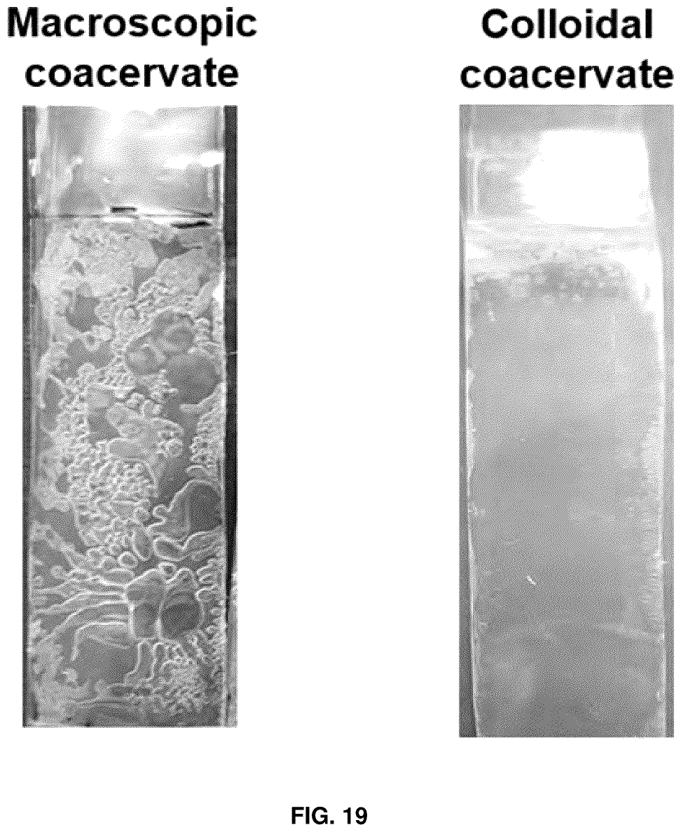

The controlled coalescence of colloidal coacervate dispersions generates uniform tie layer films, as shown in FIG. 8B. The colloidal dispersion formed by mixing a diluted PAH solution containing NaNO₃ with a diluted fatty acid solution containing NaNO₃ was deposited on an LDPE substrate, and dried in an oven at 50° C. to successfully form a continuous coacervate film. The coating uniformity was significantly improved, and, when this coating is used as a tie layer (by covering the partially dried coacervate layer with PET, compressing the resulting multilayer, and then fully drying it at 50° C.), the coating provides a large reduction in the air bubbles within the tie layer and better control over the tie layer thickness (FIG. 19).

The uniform multilayer shown in FIG. 19 was prepared as follows. Multilayer lamination was generated through the deposition of colloidal coacervate dispersions. The colloidal dispersion (0.119 ml/cm²) was pipetted onto LDPE sheets, whose edges were covered with Scotch Super 33+Electrical Tape (McMaster-Carr; Santa Fe Springs, CA) to confine the spread of the adhesive. The dispersions were then dried under ambient (22-25° C. and 50-65% RH) conditions for about 40 min, until 60-70% of the ethanol evaporated (which coincided with most of the coacervate being deposited onto the LDPE). The PET sheets were then applied on top of the coacervate, whereupon the resulting PET/coacervate/LDPE multilayers were compressed with an ISILER 150 Roller Pasta Maker (Shenzhen Deling Technology Co.; Shenzhen, China) set to a 0.35-mm sheet thickness. Wooden sticks (1.5″×9″) were then placed on top of the multilayers and compressed with a 340-g disk-shaped weight, after which the multilayers were (without removing their compression) placed into a 50° C. oven and left there overnight. Finally, each multilayer was compressed again with the pasta maker the next day and placed back in the oven with the same compression protocol, where the multilayer samples were left to dry until their weights stabilized (indicating that there was no more detectable solvent evaporation).

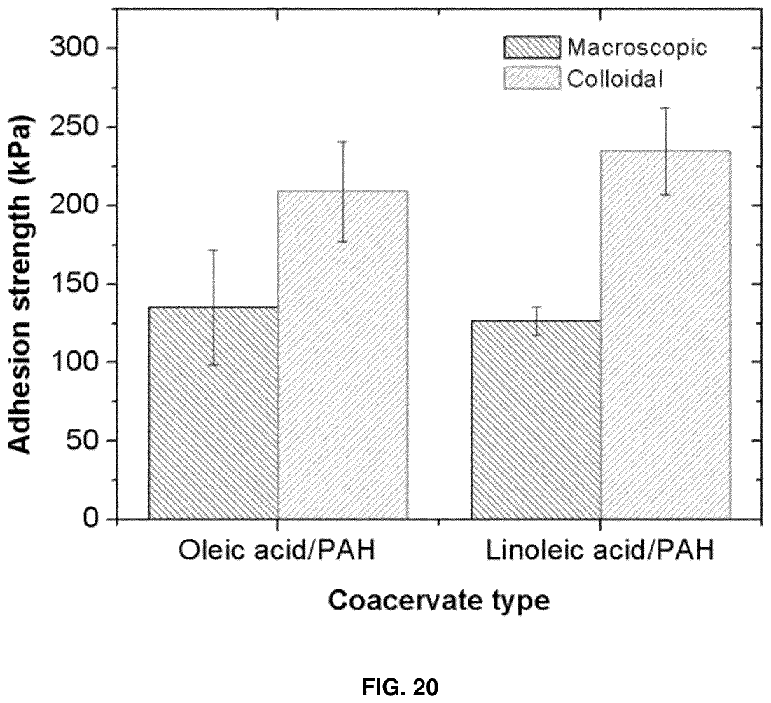

The deposition of a colloidal coacervate enables the formation of thinner and stronger-adhering tie layers (FIG. 20). As seen in FIG. 20, the mean lap shear adhesion strength for macroscopic coacervate deposition was 120-140 kPa with a tie layer thickness of 15-25 mil, whereas the mean lap shear adhesion strength for colloidal coacervate deposition was 210-240 kPa with a tie layer thickness of 4.0-4.5 mil.

### Barrier Properties

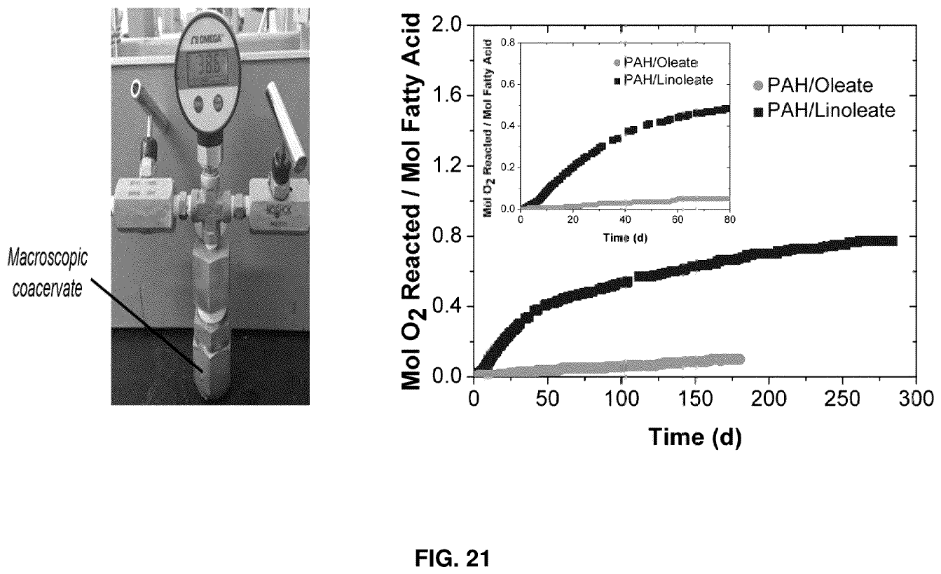

As shown in FIG. 21, dry linoleic acid/PAH coacervate slowly reacts with oxygen and can contribute to oxygen barrier properties. One mole of oxygen reacts with one mole of C═C. and linoleic acid has two such double bonds. Thus, linoleic acid/PAH coacervate compositions are useful for scavenging oxygen.

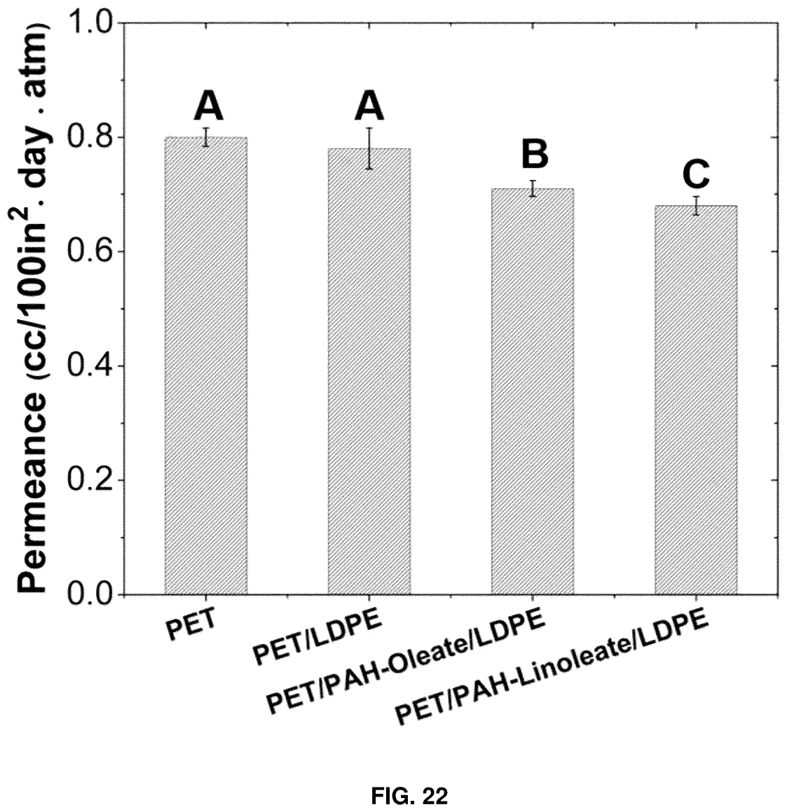

To explore whether the oxygen scavenging ability extended to improved oxygen barrier properties, oxygen permeance of PET/coacervate/LDPE, multilayer films (prepared using colloidally deposited coacervates) was analyzed and compared with that of coacervate-free PET and LDPE. As shown in FIG. 22, oxygen permeances across the (266±4-μm-thick/10.5±0.1-mil) single-layer PET films and coacervate-free PET/LDPE bilayers were similar, with values near 0.8 cc/(100 in²·day·atm). This reflected LDPE (whose layer was 190±5 μm/7.5±0.2 mil thick) being a poor oxygen barrier and virtually all resistance against oxygen transport coming from the PET layer. This reflected LDPE (whose layer was 190±5 μm/7.5±0.2 mil thick) being a poor oxygen barrier and virtually all resistance against oxygen transport coming from the PET layer. Conversely, when PAH/oleate and PAH/linoleate coacervates were used as tie-layers for the PET/LDPE films, there was a modest-yet statistically significant-reduction in the oxygen permeance (by around 11% and 14% of the permeance of the coacervate-free PET/LDPE films) to values of 0.68±0.01 and 0.71±0.01 cc/(100 in²·day·atm), respectively (FIG. 22).

### Disintegration

The disintegration (i.e., dissolution or dispersion) of the coacervate tie layers in NaOH solution leads to plastic layer delamination. PET/tie layer/LDPE multilayers were separated with 1 M NaOH at 100° C. (FIG. 9). One M NaOH at 100° C. fully dissolved the coacervate tie layer. Delamination and density-based separation occur at NaOH concentrations used in conventional recycling processes. It was observed that the delamination rates can sometimes depend on coacervate composition (FIGS. 12A-12B). Experimental multilayer packaging fragments with roughly 4-7 mm dimensions simulated the size of plastic fragments in recycling processes. The multilayer packaging delamination begins after an induction period. Depending on the fatty acid/PAH complexes, complete delamination occurs within either minutes or tens of minutes.

### Comparison to Polyelectrolytes/Ionic Surfactant/Nonionic Surfactant Mixtures

An established approach to preparing surfactant/polyelectrolyte coacervates with liquid-like properties is mixing polyelectrolytes with a mixture of an oppositely charged surfactant and a nonionic surfactant at high monovalent salt concentrations. To compare these traditional coacervates to the fatty acid/polyelectrolyte coacervates disclosed herein, coacervates were formed by complexing the ionic/nonionic surfactant mixture of Triton X-100 (TX100) and SDS with poly(diallyldimethylammonium chloride) (PDADMAC) in aqueous 0.4 M NaCl solutions. FIG. 23 shows photographs of the resulting coacervates. The coacervates were prepared at different Y^{b} values, defined by the following equation:

$${Y}^{b} = \frac{[ SDS ]}{[ SDS ] + [ TX 100 ]}$$

Mixtures prepared at a Y^{b} of 0.35 and 0.37 were centrifugated to sediment the coacervate, resulting in an oily liquid phase at the bottom. Though these coacervates could be scooped up with a spatula, they (unlike the PAH/fatty acid coacervates) had no discernable adhesive properties and fell off the spatula unless the spatula was moved very slowly.

### Peel Testing

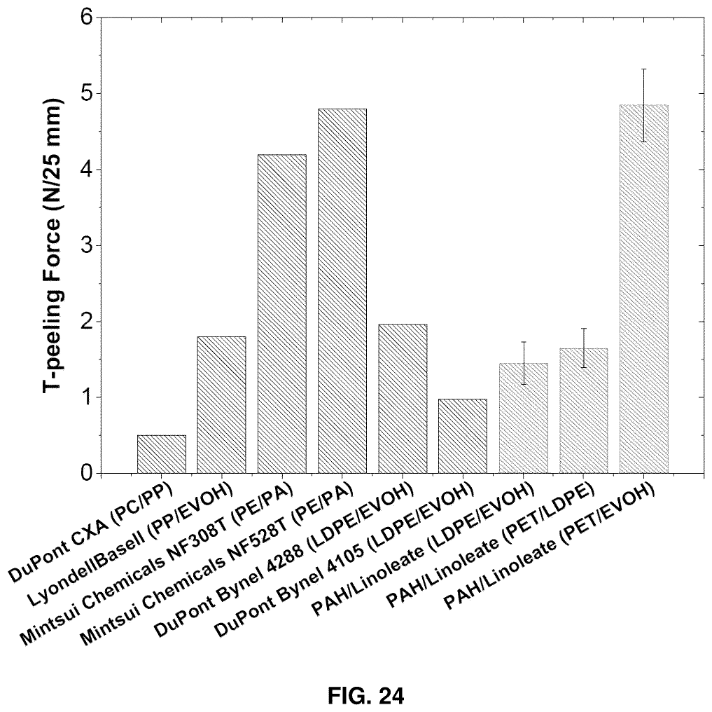

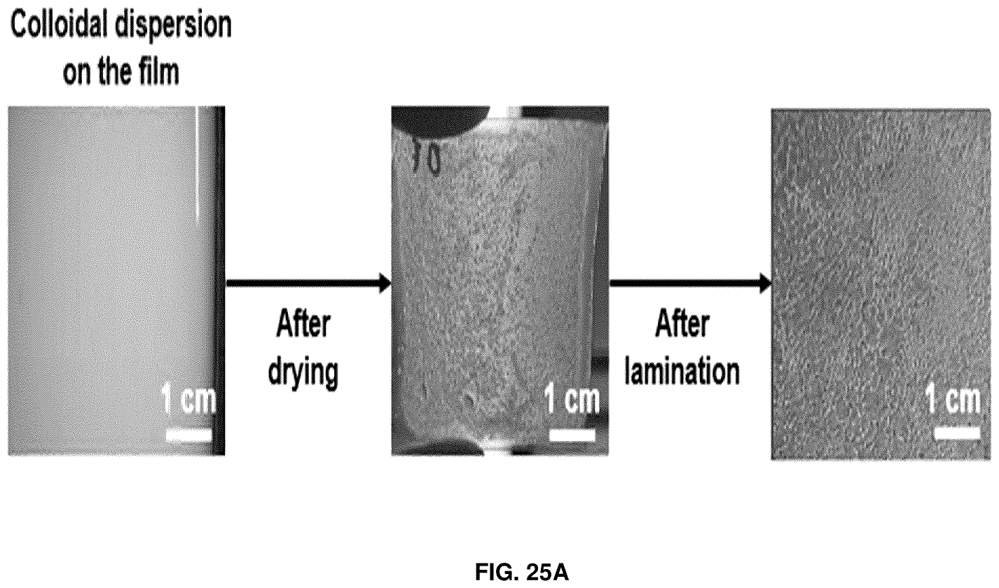

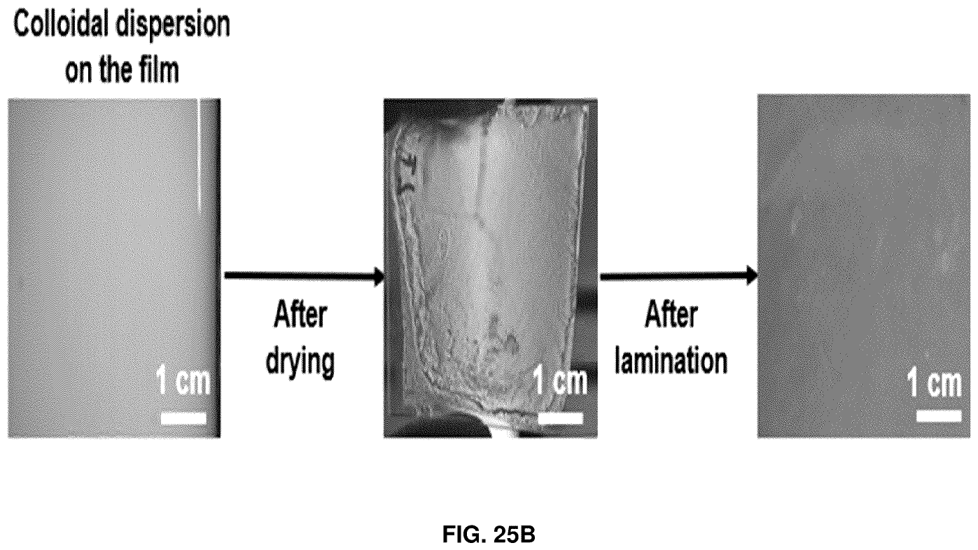

While lap-shear tests are commonly used for characterizing adhesives, the T-peeling test, where the two bonded adherend layers are peeled apart, is a workhorse technique for characterizing bond strengths achieved with tie layers. Specifically, this technique determines the relative peel resistance of the adhesive bonds between flexible layers, such as those present in multilayer films. To this end, further characterization of the PAH/oleate and PAH/linoleate coacervate adhesion strength was performed via the T-peeling test, which was performed for the three different substrate layer combinations (namely, PET/LDPE, PET/EVOH, and LDPE/EVOH). The ethanolic PAH/linoleate coacervate provided an average peeling strength of 1.5 N/25 mm for the LDPE/EVOH combination, 1.7 N/25 mm for the PET/LDPE combination, and 4.8 N/25 mm for the PET/EVOH combination (see FIG. 24). Additionally, the PET/LDPE combination was also tested with the ethanolic PAH/oleate coacervate tie layer, which generated an average peeling strength of 0.9 N/25 mm. The PAH/oleate coacervate was only tested with this layer combination because the T-peeling test showed that this coacervate produced lower adhesion strengths than the PAH/linoleate coacervate. This may have stemmed from the increased defect/air bubble formation seen upon the colloidal deposition of the PAH/oleate coacervate compared to the PAH/linoleate coacervate. When the colloidal dispersion was placed onto a (PET) polymer film and its solvent was allowed to dry, for instance, the coacervate deposited nonuniformly, accumulating coacervate at the edges and forming voids which persisted after the coacervate layer was laminated and compressed with the second (LDPE) polymer film (see FIG. 25A). In contrast, when colloidal PAH/linoleate coacervate dispersions dried, continuous coacervate films with far fewer defects formed, and after the lamination and compression between the rollers, these defects (especially the accumulation of the coacervate at the edges) disappeared (FIG. 25B). Therefore, the rest of the plastic substrate combinations were examined using the less defect-prone PAH/linoleate coacervate.

Importantly, the T-peeling strength values achieved with the coacervate tie layers were comparable to (i.e., on the same order as) values reported for commercial tie layers (see FIG. 24). Mitsui Chemicals America, Inc., for instance, developed tie layers such as ADMER™ NF528T (an anhydride-modified linear LDPE-based adhesive resin, primarily used to bond PE and EVOH during co-extrusion blown film applications), which provides a peel adhesion strength of 4.8 N/25 mm. Another tie layer developed by the same company is ADMER™ NF308T (also an anhydride-modified linear LDPE-based adhesive resin), used to bond PE and polyamide (PA) together in co-extrusion blown film applications, which provides a peel adhesion strength of 4.20 N/25 mm. Similarly, DuPont Packaging has developed a variety of tie layer products, such as the DuPont Bynel family of extrudable adhesives, such as (e.g., Bynel 4105 or Bynel 4288), which are used for laminating PE with EVOH and provide reported lamination strengths of 0.98 N/25 mm and 1.96 N/25 mm, respectively. Additionally, DuPont has developed adhesives for laminating polycarbonate (PC) with polypropylene (PP), such as the adhesive resin CXA 3101, which provides a peeling strength of 0.49 N/25 mm. Finally, LyondellBasell has developed polypropylene-based tic layer adhesives for the lamination of dissimilar materials between PP and EVOH. The highest peel strength value of their tie layer is 1.80 N/25 mm. Overall, though the substrates used with each of these commercial adhesives varied, the 1.5-4.8 N/25 mm peeling strengths achieved PAH/linoleate coacervate tie layers are well within the range of these commercial tie layer values.

### On-Demand Coacervate and MLP Dissociation

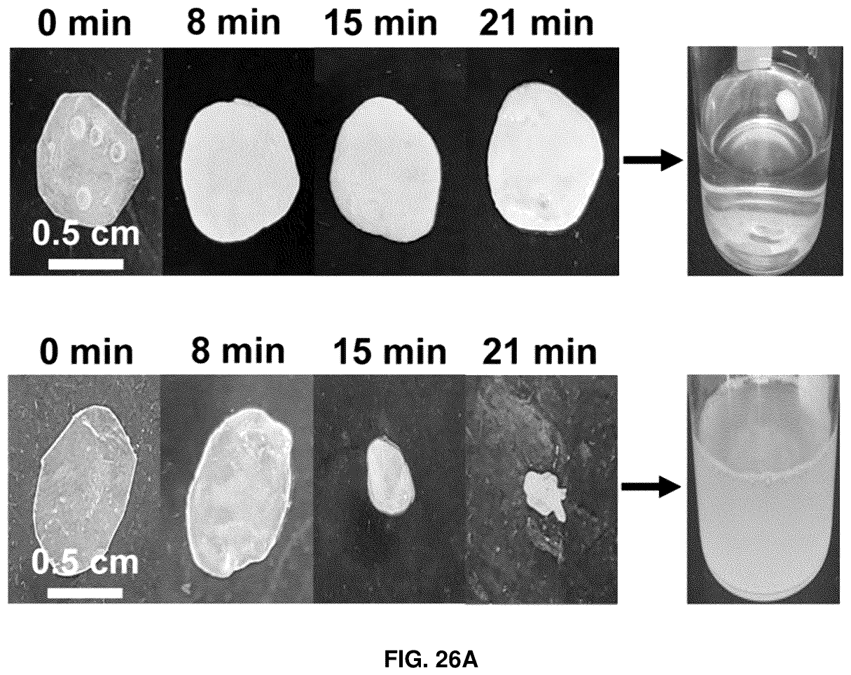

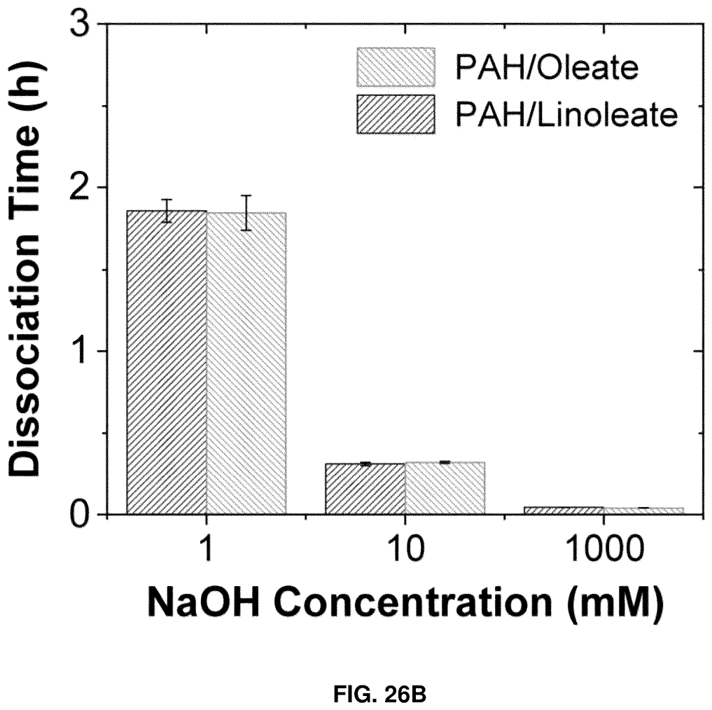

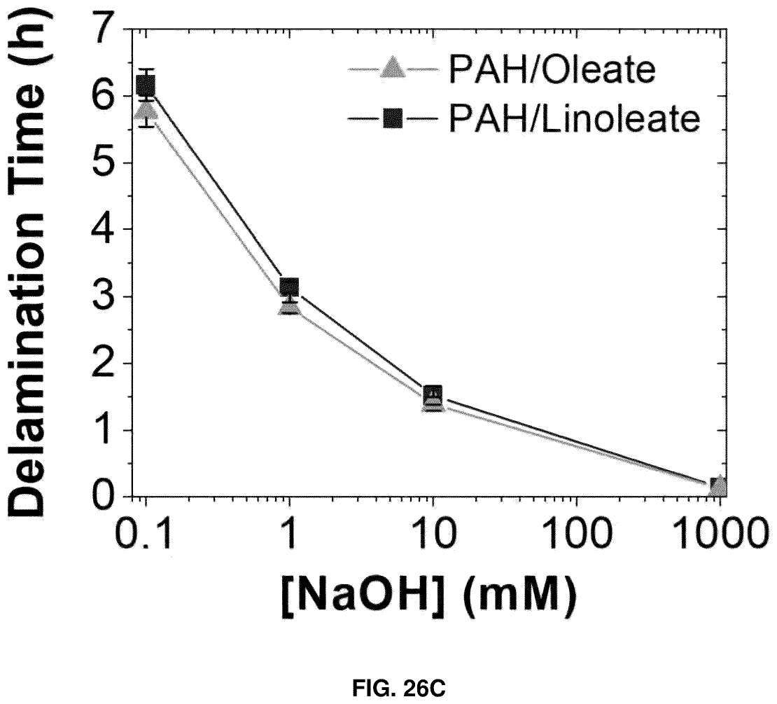

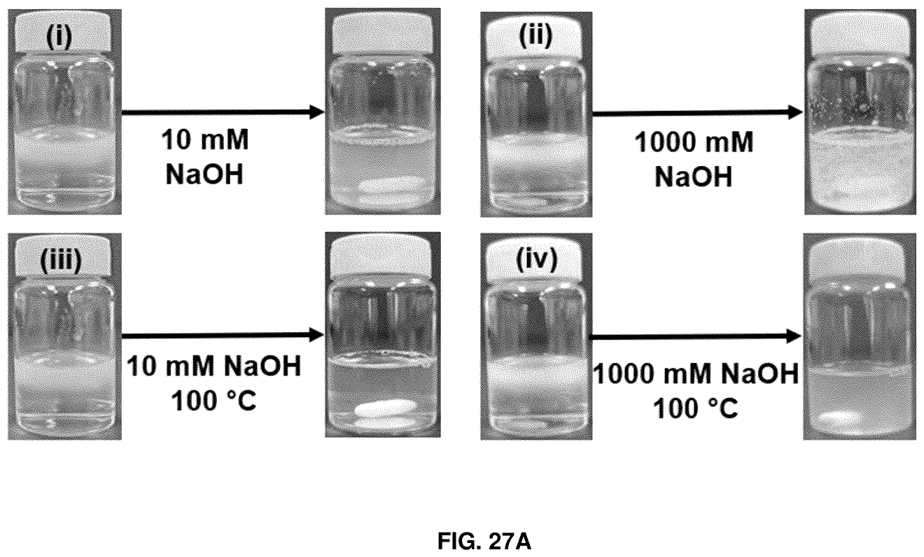

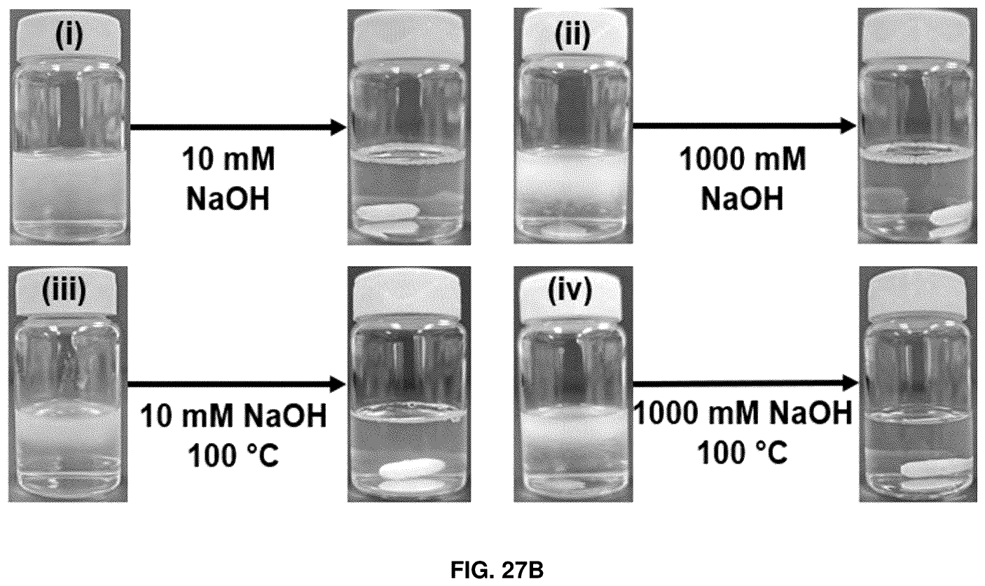

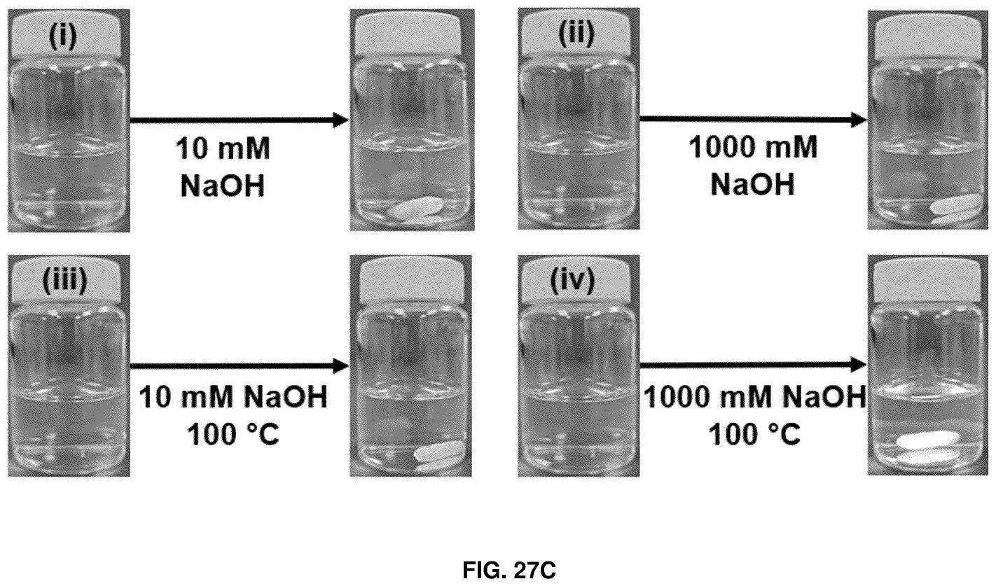

The PAH/oleate and PAH/linoleate tie layers can be dissolved on demand during commonly used recycling processes by triggering their dissociation through a pH change. Aqueous sodium hydroxide (NaOH) solutions are widely used in recycling processes for either washing away organic contaminants (at 25-1000 mM concentrations) or the alkaline hydrolysis of PET (at 1000-5000 mM concentrations). The high pH of these solutions can also lead to the deprotonation of PAH (whose effective pKₐ≈8.5) and has been used to dissolve its ionic complexes with oppositely charged molecules. To evaluate whether this effect can also be achieved with PAH/oleate and PAH/linoleate coacervates, these coacervates (prepared at the 1:1 fatty acid: PAH amine group molar ratio) were exposed to 0.1-1000 mM NaOH solutions at either room temperature or 100° C. At the lower, 0.1-100 mM NaOH concentrations and room temperature, neither coacervate dissociated. Conversely, at the higher, 1000 mM NaOH concentration, the PAH/oleate coacervate remained stable, with the only visible changes being an initial increase in its opacity and some water adsorption (and no further changes occurring after 21 min; see FIG. 26A), while the PAH/linoleate coacervate largely dispersed within 21 min (FIG. 26A). Despite breaking down, however, the coacervate did not fully dissolve and left behind a turbid dispersion (see the last image in FIG. 26A). This inability to fully dissolve the coacervates at room temperature likely reflected (1) a complexation-triggered increase in the effective PAH pKₐ, and (2) a salting out of the fatty acids by NaOH. While the effective pKₐ of ionically complexed PAH amine groups can be significantly (≥2 pH units) higher than their solution pKₐ (≈8.5), which can cause the ionic PAH complexes to remain intact up to pH-levels as high as 11-12, it is likely that the pH of the 1000 mM NaOH solutions (i.e., pH 14) was sufficient to largely deprotonate the PAH. Thus, the persistence of insoluble material in 1000 mM NaOH pointed to a salting out effect (caused by the high ionic strengths of these concentrated NaOH solutions), which was stronger for the oleic acid than for the linoleic acid (see FIGS. 27A-27B) and imperceptible through visual observation for PAH (FIG. 27C).

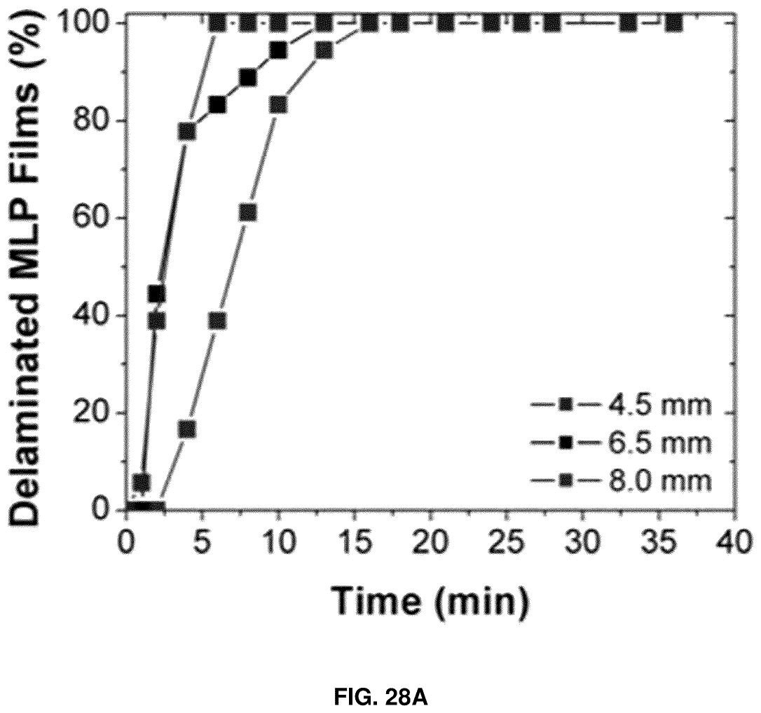

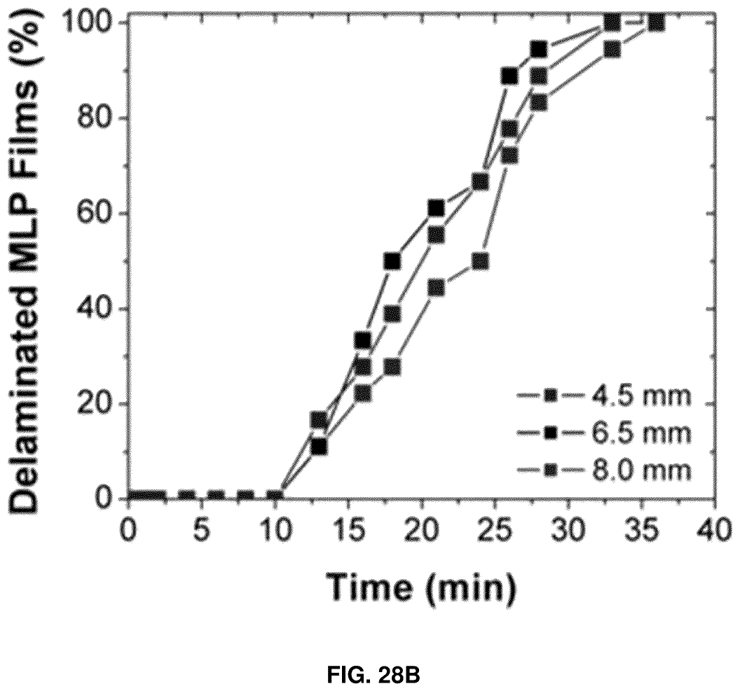

In contrast, when the NaOH solution was heated to 100° C., the PAH/linoleate coacervate dissociated in 10 mM and 1000 mM NaOH solutions after average stirring times of 0.31 h and 0.04 h, respectively (FIG. 26B). In 1 mM NaOH, the PAH/linoleate coacervate disintegrated, more slowly, after an average time of 1.83 h; however, this time produced a dispersion rather than fully dissolving, and in the 0.1 mM NaOH solution, the macroscopic coacervate samples remained intact. Similarly, the PAH/oleate coacervate disintegrated in the heated 1-1000 mM NaOH solutions after the same average stirring times as the PAH/linoleate coacervate (FIG. 26B), but remained intact at the lowest, 0.1 mM NaOH concentration. In each case where they disintegrated, however, the PAH/oleate coacervates left behind turbid colloidal dispersions (in contrast to the PAH/linoleate coacervates which appeared to fully dissolve in the heated 10-1000 mM NaOH). These findings indicate that at this temperature (1) NaOH concentrations ≥1 mM are important for substantially deprotonating the fatty acid-complexed PAH amine groups, (2) the sodium oleate, sodium linoleate, and PAH were either soluble or dispersible at even the 1000 mM NaOH concentration, and (3) that at 100° C., PAH/oleate and PAH/linoleate coacervates could be dissociated (or at least dispersed) within minutes at NaOH concentrations used during recycling. Building on these findings, delaminating the disk-shaped PET/coacervate/LDPE multilayers prepared from macroscopic coacervates was attempted. When these multilayers were placed in 1000 mM NaOH solutions (where the coacervates dissociated), the PET and LDPE layers all delaminated and separated based on their different densities (with LDPE floating and PET sinking; see FIG. 9) within ˜30 min (see FIGS. 28A-28B).

The same delamination was also achieved with the thinner PET/coacervate/LDPE multilayers prepared through colloidal deposition, which were cut into square fragments with variable (4.0 mm, 5.7 mm, and 7.1 mm) dimensions, representative of plastic fragments present during the washing stages of recycling processes. When placed in 1000 mM NaOH at 100° C., the multilayer fragments fully delaminated-regardless of their size or whether they were prepared using PAH/oleate or PAH/linoleate coacervates-within 30 min. Yet, smaller plastic fragments generally delaminated faster than their larger counterparts, and this fragment size dependence was sensitive to the coacervate type used (cf. FIGS. 12A-12B). When the PET and LDPE were laminated with the PAH/oleate coacervate, the first multilayer delamination always occurred after a 2-min induction time, with the 4.0, 5.7, and 7.1-mm fragments fully delaminating after 8, 13, and 18 min, respectively (FIG. 12A). In contrast, while the times required for the first PAH/linoleate coacervate-based 4.0 and 5.7-mm films to start delaminating were also 2 min, this time increased 4 min for 7.1-mm films (FIG. 12B). The times required to delaminate all PAH/linoleate-based films were even more fragment-size-dependent, and these varied from within 10 min for the 4.0-mm fragments to roughly 25 min for the 7.1-mm fragments.

The fragment size dependence seen with both coacervate types pointed to transport limitations (associated with water and NaOH transport into the dried coacervate tic layers) playing a role in the delamination process, which has also been reported where organic solvent (DMSO) or acid (formic acid) were used to dissolve polyurethane tie layers. In those instances, the solvent penetrated/diffused through the polymer films to dissolve the interlayer adhesive. Conversely, in the NaOH-driven disassembly in the present examples, the delamination appeared to start at the fragment edges, which allowed it to reach the bonding agent more quickly and promote the delamination from several sides. The slower and more variable delamination exhibited by the PAH/linoleate-based multilayers, on the other hand, may have reflected the stronger adhesion and more defect-free lamination achieved with these films compared to those laminated with the PAH/oleate tic layer (see FIGS. 25A-25B). Despite this variability in the delamination rates, it is clear that multilayer plastics assembled using PAH/oleate and PAH/linoleate tic layers can disassemble on demand under aqueous (i.e., organic solvent-free) conditions commonly used in recycling processes, and that this disassembly can be further accelerated by reducing film fragment size.

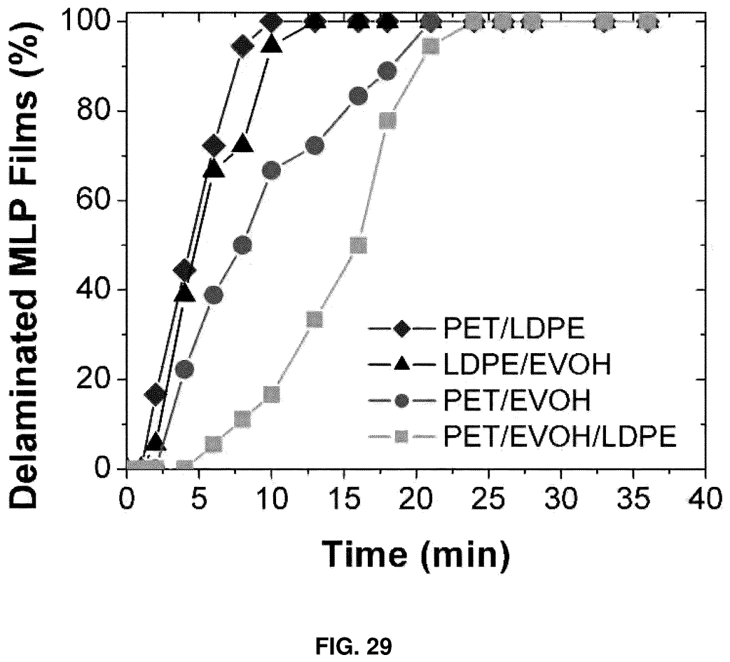

To confirm that this delamination behavior extended to other plastic types, the delamination of three-layer LDPE/coacervate/EVOH and PET/coacervate/EVOH, and five-layer PET/coacervate/EVOH/coacervate/LDPE square film fragments (with 4.0-mm dimensions) prepared using the colloidal dispersion-based lamination with the PAH/linoleate coacervate. The delamination of the LDPE/coacervate/EVOH films was similar to the PET/coacervate/LDPE multilayers, with the multilayer film samples completely separated after ˜ 12 min (FIG. 29). These similar delamination rates correlated with the nearly matching lamination strengths achieved with these multilayers (FIG. 24). The delamination of PET/coacervate/EVOH multilayer, however, required around 20 min. This slower delamination time may again be related to the relative peeling strength of this multilayer, which was almost three times higher than those of the other two combinations (FIG. 24).

Additionally, there were slightly longer induction and delamination times for the five-layer PET/coacervate/EVOH/coacervate/LDPE five-layer film, which required around 25 min to fully separate. This may have been because the tie layer thickness in these multilayers was 12 μm thicker than that in the three-layer films due to the wider roller gap setting that was used to accommodate and compress the five-layer films, as the thinnest, 0.35-mm gap used for the three-layer films was not wide enough, and the next available roller gap was 0.65 mm. Being significantly wider, this roller gap setting resulted in thicker tic layers, which may have taken longer to disintegrate. Nonetheless, it can be concluded that a 1000 mM NaOH solution at 100° C. can separate diverse MLP layers (exemplified here by PET, LDPE, and EVOH) when they are laminated with either PAH/oleate and/or PAH/linoleate coacervate tic layers.

While the ability to delaminate the multilayer films in hot 1000 mM NaOH solution enables the delamination of the component plastics under conditions used in standard mechanical recycling processes, whether this delamination could be achieved in 100° C. aqueous solutions with lower (0.1-10 mM) NaOH concentrations was also evaluated. Given the disintegration of pure coacervates in hot 1-1000 mM NaOH solutions (see FIG. 26B), both coacervates within PET/coacervate/LDPE multilayers (again prepared via colloidal coacervate deposition) disintegrated in 1 and 10 mM NaOH solutions, causing the films to delaminate. This delamination, however, was much slower than that in 1000 mM NaOH, requiring over 1 h (as opposed to 8-10 min) in 10 mM NaOH solution and close to 3 h in 1 mM NaOH solution (FIG. 26C). Moreover, the coacervate dissociation was slowed by its incorporation in the multilayer films, taking almost double the time to disintegrate the coacervates (cf. FIG. 26B and FIG. 26C). The lowest, 0.1 mM NaOH concentration did not disintegrate the coacervate, but (by weakening the adhesion to LDPE) still separated the PET from the LDPE, with detached LDPE layers floating on the top of the NaOH solution and the coacervate-bound PET layers sinking to the bottom. The time taken for this separation, however, was even longer (close to 6 h; FIG. 26C), and NaOH-free, 100° C. water failed to delaminate the films even after 8 h. Overall, PAH/oleate and PAH/linoleate coacervates provide adhesive properties that enable lamination of two dissimilar materials, (e.g., PET and LDPE) into single multilayer structures that can be disintegrated on demand in 100° C. NaOH solution, delaminating the multilayer films and separating the polymers by a difference in density.

### Further Observations on the Delamination of MLPs Prepared Using Macroscopic Coacervates

The delamination process was first tested with three different diameter disks (4.5 mm, 6.5 mm, 8.0 mm), where the macroscopic, PAH/oleate, and PAH/linoleate coacervates were used to laminate PET to LDPE. FIGS. 28A-28B show the dissociation kinetics of the PET/coacervate/LDPE films with different diameters. The PAH/oleate coacervate (FIG. 28A) produced shorter induction periods of around 2 min where nothing delaminated and took around 10 min to completely delaminate all the disks present in the solution. The PAH/linoleate coacervate (FIG. 28B), however, had an induction time of around 10 min before the multilayer fragments began delaminating, and around 30 min were needed to delaminate all the disks present in the solution. Additionally, there was a clear difference in the delamination time among the different disk sizes, being the slowest for the largest disks (of 8 mm diameter) and the fastest for the smallest (4.5 mm) disks. This was because the adhesive started to disintegrate from the edges that were directly exposed to the NaOH solution where, as the solution dissolved the adhesive at the edges, the layers started to separate, whereupon the NaOH solution could reach more of the adhesive. Thus, the greater the lamination area, the longer the delamination time of the disk.

### Summary

Complex coacervation enables adhesive/tie layer development. Lamination of dissimilar plastic layers, and dissolution and delamination under readily accessible recycling conditions have been achieved. Applying the coacervate through colloidal deposition improves the film formation. The tie layer may also act as an oxygen scavenging material.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A coacervate composition comprising:
a complex between a polyelectrolyte and a surfactant; and
a solvent;
wherein the surfactant comprises a cis double bond and a hydrophobic tail; and
wherein the complex is moldable, adhesive, viscoelastic, or a viscous liquid having a viscosity that exceeds a viscosity of the solvent.

2. The coacervate composition of claim 1, wherein the cis double bond is a non-terminal double bond.

3. The coacervate composition of claim 1, wherein the surfactant is a fatty acid.

4. The coacervate composition of claim 1, wherein the polyelectrolyte comprises polyallylamine (PAH).

5. The coacervate composition of claim 1, wherein the polyelectrolyte comprises polyallylamine (PAH) and the surfactant comprises an unsaturated fatty acid.

6. The coacervate composition of claim 1, wherein the polyelectrolyte comprises polyallylamine (PAH) and the surfactant comprises linoleic acid or oleic acid.

7. The coacervate composition of claim 6, wherein the surfactant comprises linoleic acid and the solvent comprises water, ethanol, methanol, or isopropanol.

8. The coacervate composition of claim 6, wherein the surfactant comprises oleic acid and the solvent comprises water or ethanol.

9. The coacervate composition of claim 1, further comprising an electrolyte soluble in the solvent, wherein the electrolyte is an alkali metal salt, a halide salt, an ammonium salt, a nitrate, a sulfate, or a phosphate.

10. The coacervate composition of claim 1, wherein the coacervate composition is in the form of a uniform or homogeneous film disposed on a plastic support.

11. The coacervate composition of claim 1, further comprising a payload, wherein the coacervate composition is configured to release the payload over time, wherein the payload comprises a drug, a fragrance, or a dye.

12. The coacervate composition of claim 1, further comprising a second surfactant, wherein the surfactant is a nonionic surfactant and the second surfactant is an ionic surfactant.

13. A multilayer packaging material comprising:
a first plastic layer comprising a first plastic material;
a tie layer comprising the coacervate composition of claim 1; and
a second plastic layer comprising a second plastic material;
wherein the tie layer is laminated together with the first plastic layer and the second plastic layer.

14. The multilayer packaging material of claim 13, wherein the first plastic material or the second plastic material comprises PET, LDPE, EVOH, nylon, or poly(ethylene-vinyl acetate) (PEVA).

15. A multilayer packaging material comprising:
a first plastic layer comprising a first plastic material;
a first tie layer comprising a first coacervate composition, wherein the first coacervate composition comprises a first complex between a first polyelectrolyte and a first surfactant having a cis double bond;
a second plastic layer comprising a second plastic material, wherein the first tie layer connects the first plastic layer and the second plastic layer;
a second tie layer comprising a second coacervate composition, wherein the second coacervate composition comprises a second complex between a second polyelectrolyte and a second surfactant having a cis double bond; and
a third plastic layer comprising a third plastic material, wherein the second tie layer connects the second plastic layer and the third plastic layer.

16. The multilayer packaging material of claim 15, wherein one or both of the first polyelectrolyte and the second polyelectrolyte comprises PAH, and one or both of the first surfactant and the second surfactant comprises oleic acid or linoleic acid.

17. A method of recycling a multilayer packaging material, the method comprising dissolving the tie layer of the multilayer packaging material of claim 13 in a basic solution at a temperature and for a period of time sufficient to completely dissolve the tie layer.

18. The method of claim 17, wherein the basic solution has a room-temperature pH of at least about 11-12.

19. A method for making a coacervate composition, the method comprising:
forming a complex between a polyelectrolyte and a surfactant having a cis double bond and a hydrophobic tail; and
solvating the complex in a solvent to form a coacervate composition, wherein the complex is moldable, adhesive, viscoelastic, or a viscous liquid having a viscosity exceeding a viscosity of the solvent.

20. The method of claim 19, wherein the solvent is water or an alcohol, or a mixture of water and one or more alcohols.
